(19) United States
(12) Patent Application Publication
(10) Pub. No.: US 2026/0335406 A1
(43) Pub. Date: Oct. 29, 2026

(54) MAJOR HISTOCOMPATIBILITY COMPLEX (MHC) COMPOSITIONS AND METHODS OF USE THEREOF

(71) Applicant: Centivax, Inc., South San Francisco, CA (US)

(72) Inventors: Sawsan Youssef, Menlo Park, CA (US); Jacob Glanville, San Francisco, CA (US)

(21) Appl. No.: 19/393,928

(22) Filed: Nov. 19, 2025

Related U.S. Application Data
(63) Continuation of application No. 19/065,292, filed on Feb. 27, 2025, now abandoned.
Application No. 19/065,292 is a continuation of application No. 18/777,029, filed on Jul. 18, 2024, now abandoned.
Application No. 18/777,029 is a continuation of application No. 18/537,445, filed on Dec. 12, 2023, now abandoned.
Application No. 18/537,445 is a continuation of application No. 16/899,512, filed on Jun. 11, 2020, now abandoned.
Application No. 16/899,512 is a continuation of application No. PCT/US2018/067380, filed on Dec. 21, 2018.
(60) Provisional application No. 62/609,589, filed on Dec. 22, 2017.

(51) Int. Cl. A61K 38/17; A61K 48/00; A61P 35/00; C07K 14/705

(52) U.S. Cl. CPC A61K 38/1774; A61K 48/005; A61P 35/00; C07K 14/70539; C12N 2710/10343; C12N 2710/10371

(57) ABSTRACT

Immunotherapeutic compositions including class I MHC component, non-classical MHC class I component, or class II MHC components and methods of use thereof are described. The class I MHC, non-classical class I MHC, class II MHC components can be non-naturally occurring MHC component. Additionally, immunotherapeutic compositions comprising a nucleic acid encoding a deactivated CRISPR-associated nuclease fused to a TET enzyme and a gRNA targeting methylated regions of genetic elements controlling expression of MHC genes and method of use thereof are described. The compositions and methods described herein can further comprise administration of the immunotherapeutic composition with an immune checkpoint inhibitor.

### RELATED APPLICATIONS

This application is a continuation application of U.S. patent application Ser. No. 19/065,292, filed Feb. 27, 2025, which is a continuation application of U.S. patent application Ser. No. 18/777,029, filed Jul. 18, 2024, which is a continuation application of U.S. patent application Ser. No. 18/537,445, filed Dec. 12, 2023, which is a continuation application of U.S. patent application Ser. No. 16/899,512, filed Jun. 11, 2020, which is a continuation application of International Patent Application No. PCT/US2018/067380, filed Dec. 21, 2018, which claims the benefit of U.S. Provisional Application No. 62/609,589, filed Dec. 22, 2017, each of which is entirely incorporated herein by reference.

### BACKGROUND OF THE DISCLOSURE

Major histocompatibility complex (MHC) molecules are important in the immune response of the body as they bind to antigens derived from pathogens or tumors, displaying them on the cell surface for recognition by T-cells. Genes in the MHC, often referred to in humans as human leukocyte antigen (HLA) genes, include class I, class II MHC, non-classical MHC I, and non-classical MHC II genes. Class I MHC molecules are ubiquitously expressed on the surfaces of adult somatic cells and usually present peptides of cytosolic origin, although through mechanisms of cross-presentation they can present extracellular antigens. Non-classical MHC I molecules can be recognized by natural killer (NK) cells and CD8⁺ T cells. Class II MHC molecules bind to peptides derived from proteins degraded in the endocytic pathway and are usually restricted to professional antigen presenting cells (APCs), such as dendritic cells, macrophages, and B cells, however, expression of MHC class II molecules can be induced in other types of cells, such as tumor cells. Non-classical MHC II molecules are generally not exposed on cell surface, but exposed on internal membranes in lysosomes.

One way tumor cells avoid recognition by T-cells is to express immune checkpoints, masking their identity as cancerous cells and evading immune system attack. Immune checkpoint inhibitors have been used to block this method of action and allow T-cells to recognize these cells as cancerous. However, these therapies have proven ineffective in some cancers.

Immune checkpoint inhibitors can only be effective if the T-cell is first able recognize a tumor cell. Some cancers have been shown to lack or significantly reduce expression of MHC molecules which can interfere with this tumor recognition, and could be a way in which tumor cells avoid detection. Therefore, it is desirable to develop methods of increasing the expression of MHC in cancer cells, as this could increase not only the innate immune response of the body in absence of any additional therapies but may also serve as a way to enhance the effectiveness of therapeutic agents, such as immune checkpoint inhibitors, in previously unresponsive cancers.

### SUMMARY OF THE DISCLOSURE

Provided herein are immunotherapeutic compositions, comprising a nucleic acid molecule encoding a MHC component or a fragment thereof. The MHC component can be formulated with at least one, two, three, four or more different excipients for delivery to a subject or an individual. The MHC component can be a naturally occurring MHC component, or alternatively the MHC component can be non-naturally occurring. In some embodiments, the MHC component is non-naturally occurring and shows enhanced recognition by a T cell relative to a naturally occurring MHC component. In some embodiments, the MHC component is naturally occurring, and a cell expressing the heterologous MHC component has an enhanced recognition by a T cell relative to a similar cell not modified to express the heterologous MHC component. In some instances the modified cell is a cancer cell. Such cancer cell can be a solid tumor cancer cell. Such cancer cell can be a breast cancer cell, a prostate cancer cell, a lung cancer cell, a pancreatic cancer cell, an ovarian cancer cell, a liver cancer cell, a colon cancer cell, or any other cancer cell.

In some embodiments, a nucleic acid molecule of the disclosure encodes a non-naturally occurring MHC component. A non-naturally occurring MHC component can be an engineered MHC component having a high sequence homology to a naturally occurring MHC component.

In some instances, a composition herein comprises a non-naturally occurring homolog of a naturally occurring MHC component. Such homolog can comprise at least one variant compared to a nucleic acid molecule encoding a naturally occurring MHC component. In some embodiments, the variant is a mutation, an insertion, a deletion, or a duplication. An MHC homolog herein preferably has at least 80%, 85%, 90%, 95%, 96%, 97%, 98%, 99% or 99.5% amino acid sequence homology to a naturally occurring MHC component. In some embodiments, a nucleic acid molecule is at least 80%, 90%, 95%, 98%, or 99% similar or has at least 80%, 90%, 95%, 98%, or 99% sequence homology to the nucleic acid sequence encoding the naturally occurring MHC component. In some embodiments, a nucleic acid molecule encodes an MHC component that is at least 80%, 90%, 95%, 98%, or 99% similar or has at least 80%, 90%, 95%, 98%, or 99% sequence homology to an MHC component that is naturally occurring. In some embodiments, the nucleic acid molecule is at least 80%, 90%, 95%, 98%, or 99% similar to the nucleic acid sequence encoding the naturally occurring MHC component. In some embodiments, the nucleic acid encodes an MHC component that is at least 80%, 90%, 95%, 98%, or 99% similar to a naturally occurring MHC component.

In some embodiments, the MHC component is a gene selected from the list consisting of: HLA-A, HLA-B, HLA-C, HLA-E, HLA-G, HLA-F, HLA-DRA, HLA-DRB1, HLA-DRB3, HLA-DRB4, HLA-DRB5, HLA-DQA1, HLA-DQB1, HLA-DOA, HLA-DOB, HLA-DMA, HLA-DMB, HLA-DPA1, and HLA-DPB1. The MHC component can be a class I MHC component. In some embodiments, the class I MHC component is a heavy (α) chain, a light chain (β₂ microglobulin), or a combination thereof.

In some embodiments, the immunotherapeutic composition further comprises a second nucleic acid molecule encoding a second class I MHC component or functional (e.g., antigenic) fragment thereof. In some embodiments, the second class I MHC component is a heavy (α) chain, a light chain (β₂ microglobulin), or a combination thereof. In some embodiments, the second class I MHC component is a naturally occurring or a non-naturally occurring MHC component. In some embodiments, a naturally occurring or a non-naturally occurring MHC component is a class II MHC component. In some embodiments, the class II MHC component comprises an alpha (α) chain, a beta (β) chain, or a combination thereof. In some embodiments, the immunotherapeutic composition further comprises a second nucleic acid molecule encoding a second class II MHC component or a functional fragment thereof. In some embodiments, the second class II MHC component comprises an alpha (α) chain, a beta (β) chain or a combination thereof. In some embodiments, the second class II MHC component is a naturally occurring or a non-naturally occurring MHC component. In some embodiments, the nucleic acid molecule is DNA or RNA. In some embodiments, the nucleic acid is a plasmid. In some embodiments, the nucleic acid is a viral vector. In some embodiments, the viral vector is an alphavirus, a retrovirus, an adenovirus, a herpes virus, poxvirus, lentivirus, oncolytic virus, reovirus, or an adeno associated virus (AAV). In some embodiments, the nucleic acid is formulated for targeted delivery to a tumor cell. In some embodiments, the nucleic acid is formulated in a vesicle such as a liposome, exosome, lipid nanoparticle, or a biomaterial. In some embodiments, the liposome comprises the additional therapeutic compound, a polyethylene glycol (PEG), a cell-penetrating peptide, a ligand, an aptamer, an antibody, or a combination thereof. In some embodiments, the liposome is formulated for targeted delivery to a cancer cell. In some embodiments, the method further comprises at least one pharmaceutically acceptable excipient, diluent, or carrier. In some embodiments, the method further comprises a unit dose of between about 0.01 μg to about 100 μg of the nucleic acid disclosed herein. In other embodiments, the method further comprises a unit does of between about 0.01 μg to about 100 μg of the MHC molecules encoded by the nucleic acid disclosed herein.

Also provided herein are methods for treating a cancer in an individual, comprising administering to the individual a nucleic acid molecule encoding a MHC component or a functional fragment thereof. In some embodiments, the MHC component can be non-naturally occurring. In other embodiments, the MHC component is naturally occurring. In some embodiments, the non-naturally occurring MHC component shows enhanced recognition by a T cell relative to a naturally occurring MHC component. In some embodiments, the cancer is ovarian cancer, pancreatic cancer, or colon cancer. In some embodiments, the cancer has reduced MHC expression. In some embodiments, the method further comprises determining the sequence of a native MHC component of the individual. In some embodiments, the method further comprises diagnosing the cancer with reduced MHC expression comprising: (a) obtaining a biological sample from the individual, (b) isolating cancerous cells from the biological sample; and (c) detecting whether MHC expression in the isolated cancerous cells is reduced relative to a control. In some embodiments, the individual has previously been administered an additional therapeutic compound selected from the group consisting of an immune checkpoint inhibitor, an immune checkpoint stimulator, a cancer vaccine, a small molecule therapy, a monoclonal antibody, a cytokine, a cellular therapy, or a combination thereof. In some embodiments, the method further comprises administering an additional therapeutic compound to the individual. In some embodiments, the additional therapeutic compound is an immune checkpoint inhibitor, an immune checkpoint stimulator, a cancer vaccine, a small molecule therapy, a monoclonal antibody, a cytokine, or a cellular therapy. In some embodiments, the immune checkpoint inhibitor is a molecule which binds to A2AR, B7-H3, B7-H4, BTLA, CTLA-4, IDO, KIR, LAG3, PD-1, TIM-3, VISTA, or a ligand thereof. In some embodiments, the immune checkpoint stimulator is a molecule which binds to CD27, CD28, CD40, CD122, CD137, OX40, GITR, ICOS, or a ligand thereof. In some embodiments, the small molecule therapy is a proteasome inhibitor, a tyrosine kinase inhibitor, a cyclin-dependent kinase inhibitor, or a polyADP-ribose polymerase (PARP) inhibitor. In some embodiments, the cytokine is INFα, INFβ, IFNγ, or TNF. In some embodiments, the cellular therapy is an adoptive T cell transfer (ACT) therapy. Additionally or alternatively, the cellular therapy can be chimeric antigen receptor (CAR) T-cell therapy or T-cell antigen coupler (TAC) T-cell therapy.

In some embodiments, administration of the nucleic acid molecule to the individual results in the cancer showing an increased sensitivity to the at least one additional therapeutic compound. In some embodiments, the nucleic acid molecule is a non-naturally occurring MHC component that comprises at least one variant compared to a nucleic acid molecule encoding a naturally occurring MHC component. In some embodiments, the variant is a mutation, an insertion, a deletion, or a duplication. In some embodiments, the MHC component is a gene selected from the list consisting of: HLA-A, HLA-B, HLA-C, HLA-DRA, HLA-E, HLA-G, HLA-F, HLA-DRB1, HLA-DRB3, HLA-DRB4, HLA-DRB5, HLA-DQA1, HLA-DQB1, HLA-DOA, HLA-DOB, HLA-DMA, HLA-DMB, HLA-DPA1, and HLA-DPB1. In some embodiments, the nucleic acid molecule is at least 95% similar to the nucleic acid sequence encoding the naturally occurring MHC component. In some embodiments, the nucleic acid molecule is at least 80% similar to the nucleic acid sequence encoding the naturally occurring MHC component. In some embodiments, the MHC component is a class I MHC component. In some embodiments, the class I MHC component is a heavy (α) chain, a light chain (β₂ microglobulin), or a combination thereof. In some embodiments, the immunotherapeutic composition further comprises a second nucleic acid molecule encoding a second class I MHC component or fragment thereof. In some embodiments, the second class I MHC component is a heavy (α) chain, a light chain (β₂ microglobulin), or a combination thereof. In some embodiments, the second class I MHC component is a naturally occurring or a non-naturally occurring MHC component. In some embodiments, the MHC component is a class II MHC component. In some embodiments, the class II MHC component comprises an alpha (α) chain, a beta (β) chain, or a combination thereof. In some embodiments, the immunotherapeutic composition further comprises a second nucleic acid molecule encoding a second class II MHC component or a fragment thereof. In some embodiments, the second class II MHC component comprises an alpha (α) chain, a beta (β) chain or a combination thereof. In some embodiments, the second class II MHC component is a naturally occurring or a non-naturally occurring MHC component. In some embodiments, the nucleic acid molecule is DNA or RNA. In some embodiments, the nucleic acid is a plasmid. In some embodiments, the nucleic acid is a viral vector. In some embodiments, the viral vector is an alphavirus, a retrovirus, an adenovirus, a herpes virus, poxvirus, lentivirus, oncolytic virus, reovirus, or an adeno associated virus (AAV). In some embodiments, the nucleic acid is formulated for targeted delivery to a tumor cell. In some embodiments, the nucleic acid is formulated in a liposome. In some embodiments, the liposome comprises the additional therapeutic compound, a polyethylene glycol (PEG), a cell-penetrating peptide, a ligand, an aptamer, an antibody, or a combination thereof. In some embodiments, the liposome is formulated for targeted delivery to a cancer cell.

Also provided herein are immunotherapeutic compositions, comprising: a nucleic acid encoding a deactivated CRISPR-associated nuclease fused to an enzyme that modifies a nucleic acid molecule (e.g., a TET enzyme) and a guide RNA (gRNA) with a region complementary to a transcription factor or a promoter of an MHC gene. In some embodiments, the MHC gene is HLA-A, HLA-B, HLA-C, HLA-E, HLA-G, HLA-F, HLA-DRA, HLA-DRB1, HLA-DRB3, HLA-DRB4, HLA-DRB5, HLA-DQA1, HLA-DQB1, HLA-DOA, HLA-DOB, HLA-DMA, HLA-DMB, HLA-DPA1, and HLA-DPB1. In some embodiments, the deactivated CRISPR-associated nuclease is deactivated Cas9 (dCas9). In some embodiments, the TET enzyme is TET1, TET2, TET3, or a catalytic domain thereof. In some embodiments, the nucleic acid molecule is DNA or RNA. In some embodiments, the nucleic acid is a plasmid. In some embodiments, the nucleic acid is a viral vector. In some embodiments, the viral vector is an alphavirus, a retrovirus, an adenovirus, a herpes virus, poxvirus, lentivirus, oncolytic virus, reovirus, or an adeno associated virus (AAV). In some embodiments, the nucleic acid is formulated for targeted delivery to a tumor cell. In some embodiments, the nucleic acid is formulated in a liposome. In some embodiments, the liposome comprises the additional therapeutic compound, a polyethylene glycol (PEG), a cell-penetrating peptide, a ligand, an aptamer, an antibody, or a combination thereof. In some embodiments, the liposome is formulated for targeted delivery to a cancer cell. In some embodiments, the composition further comprises at least one pharmaceutically acceptable excipient, diluent, or carrier.

Also provided herein are methods for increasing expression of an MHC gene in a cancer in an individual, comprising administering to the individual an immunotherapeutic composition comprising: a nucleic acid encoding a deactivated CRISPR-associated nuclease fused to a TET enzyme and a guide RNA (gRNA) with a region complementary to a transcription factor or a promoter of the MHC gene. In some embodiments, the MHC gene is HLA-A, HLA-B, HLA-C, HLA-E, HLA-G, HLA-F, HLA-DRA, HLA-DRB1, HLA-DRB3, HLA-DRB4, HLA-DRB5, HLA-DQA1, HLA-DQB1, HLA-DOA, HLA-DOB, HLA-DMA, HLA-DMB, HLA-DPA1, and HLA-DPB1. In some embodiments, the cancer is ovarian cancer, pancreatic cancer, or colon cancer. In some embodiments, the cancer has reduced MHC expression. In some embodiments, the method further comprises diagnosing the cancer with reduced MHC expression comprising: (a) obtaining a biological sample from the individual, (b) isolating cancerous cells from the biological sample; and (c) detecting whether MHC expression in the isolated cancerous cells is reduced. In some embodiments, the individual has previously been administered an additional therapeutic compound selected from the group consisting of: an immune checkpoint inhibitor, an immune checkpoint stimulator, a cancer vaccine, a small molecule therapy, a monoclonal antibody, a cytokine, a cellular therapy, or a combination thereof. In some embodiments, the method further comprises administering an additional therapeutic compound to the individual. In some embodiments, the additional therapeutic compound is an immune checkpoint inhibitor, an immune checkpoint stimulator, a cancer vaccine, a small molecule therapy, a monoclonal antibody, a cytokine, or a cellular therapy. In some embodiments, the immune checkpoint inhibitor is a molecule which binds to A2AR, B7-H3, B7-H4, BTLA, CTLA-4, IDO, KIR, LAG3, PD-1, TIM-3, VISTA, or a ligand thereof. In some embodiments, the immune checkpoint stimulator is a molecule which binds to CD27, CD28, CD40, CD122, CD137, OX40, GITR, ICOS, or a ligand thereof. In some embodiments, the small molecule therapy is a proteasome inhibitor, a tyrosine kinase inhibitor, a cyclin-dependent kinase inhibitor, or a polyADP-ribose polymerase (PARP) inhibitor. In some embodiments, the cytokine is INFα, INFβ, IFNγ, or TNF. In some embodiments, the cellular therapy is an adoptive T cell transfer (ACT) therapy. Additionally or alternatively, the cellular therapy can be chimeric antigen receptor (CAR) T-cell therapy or T-cell antigen coupler (TAC) T-cell therapy.

In some embodiments, expression of the nucleic acid molecule by the cancer results in the cancer showing an increased sensitivity to the at least one additional therapeutic compound. In some embodiments, the deactivated CRISPR-associated nuclease is deactivated Cas9 (dCas9). In some embodiments, the TET enzyme is TET1, TET2, TET3, or a catalytic domain thereof. In some embodiments, the nucleic acid molecule is DNA or RNA. In some embodiments, the nucleic acid is a plasmid. In some embodiments, the nucleic acid is a viral vector. In some embodiments, the viral vector is an alphavirus, a retrovirus, an adenovirus, a herpes virus, poxvirus, lentivirus, oncolytic virus, reovirus, or an adeno associated virus (AAV). In some embodiments, the nucleic acid is formulated for targeted delivery to a tumor cell. In some embodiments, the nucleic acid is formulated in a liposome. In some embodiments, the liposome comprises the additional therapeutic compound, a polyethylene glycol (PEG), a cell-penetrating peptide, a ligand, an aptamer, an antibody, or a combination thereof. In some embodiments, the liposome is formulated for targeted delivery to a cancer.

Further, provided herein are immunotherapeutic compositions, comprising a nucleic acid molecule encoding a regulator of an MHC molecule. In some embodiments, the regulator of the MHC molecule is selected from the group consisting of: transactivator, a transcription factor, an acetyltransferase, a methyltransferase, an elongation factor, and any combination thereof. In some embodiments, the transactivator is selected from the group consisting of class II, major histocompatibility complex, transactivator (CIITA) and NOD-like receptor family CARD domain containing 5 (NLRC5). In some embodiments, the transcription factor is selected from the group consisting of: a nuclear transcription factor Y (NF-Y), cAMP response element-binding protein (CREB), a regulatory factor X (RFX), an interferon regulatory factor (IRF), a signal transducer and activator of transcription (STAT), a ubiquitous transcription factor (USF), and nuclear factor kappa-light-chain-enhancer of activated B cells (NF-κB). In some embodiments, wherein the NF-Y is selected from the group consisting of NF-Ya, NF-Yb, and NF-Yc. In some embodiments, the RFX is selected from the group consisting of RFXANK/RFXB, RFX5, and RFXAP. In some embodiments, the IRF is selected form the group consisting of: IRF-1, IRF-2, IRF-3, IRF-4, IRF-5, IRF-6, IRF-7, IRF-8, and IRF-9. In some embodiments, the STAT is selected from the group consisting of: STAT-1, STAT-2, STAT-3, STAT-4, STAT-5, and STAT-6. In some embodiments, the USF is selected from the group consisting of: USF-1 and USF-2. In some embodiments, the acetyltransferase is selected from the group consisting of: CREB-binding protein (CBP), p300, and p300/CBP-associated factor (pCAF). In some embodiments, the methyltransferase is Enhancer of Zeste Homolog 2 (EZH2), protein arginine N-methyltransferase 1 (PRMT1), and coactivator-associated arginine methyltransferase 1 (CARM1). In some embodiments, the elongation factor is positive transcriptional elongation factor (pTEFb). In some embodiments, the nucleic acid molecule is DNA or RNA. In some embodiments, the nucleic acid is a plasmid. In some embodiments, the nucleic acid is a viral vector. In some embodiments, the viral vector is an alphavirus, a retrovirus, an adenovirus, a herpes virus, poxvirus, lentivirus, oncolytic virus, reovirus, or an adeno associated virus (AAV). In some embodiments, the nucleic acid is formulated for targeted delivery to a tumor cell. In some embodiments, the nucleic acid is formulated in a liposome. In some embodiments, the liposome comprises the additional therapeutic compound, a polyethylene glycol (PEG), a cell-penetrating peptide, a ligand, an aptamer, an antibody, or a combination thereof. In some embodiments, the liposome is formulated for targeted delivery to a cancer cell. In some embodiments, the immunotherapeutic compositions further comprise at least one pharmaceutically acceptable excipient, diluent, or carrier.

Moreover, provided herein are methods for treating a cancer in an individual, comprising administering to the individual a nucleic acid molecule encoding a regulator of an MHC molecule. In some embodiments, the cancer is ovarian cancer, pancreatic cancer, or colon cancer. In some embodiments, the cancer has reduced MHC expression. In some embodiments, the methods further comprise diagnosing the cancer with reduced MHC expression comprising: (a) obtaining a biological sample from the individual, (b) isolating cancerous cells from the biological sample; and (c) detecting whether MHC expression in the isolated cancerous cells is reduced relative to a control. In some embodiments, the individual has previously been administered an additional therapeutic compound selected from the group consisting of: an immune checkpoint inhibitor, an immune checkpoint stimulator, a cancer vaccine, a small molecule therapy, a monoclonal antibody, a cytokine, a cellular therapy, or a combination thereof. In some embodiments, the methods further comprise administering an additional therapeutic compound to the individual. In some embodiments, the additional therapeutic compound is an immune checkpoint inhibitor, an immune checkpoint stimulator, a cancer vaccine, a small molecule therapy, a monoclonal antibody, a cytokine, or a cellular therapy. In some embodiments, the immune checkpoint inhibitor is a molecule which binds to A2AR, B7-H3, B7-H4, BTLA, CTLA-4, IDO, KIR, LAG3, PD-1, TIM-3, VISTA, or a ligand thereof. In some embodiments, the immune checkpoint stimulator is a molecule which binds to CD27, CD28, CD40, CD122, CD137, OX40, GITR, ICOS, or a ligand thereof. In some embodiments, the small molecule therapy is a proteasome inhibitor, a tyrosine kinase inhibitor, a cyclin-dependent kinase inhibitor, or a polyADP-ribose polymerase (PARP) inhibitor. In some embodiments, the cytokine is INFα, INFβ, IFNγ, or TNF. In some embodiments, the cellular therapy is an adoptive T cell transfer (ACT) therapy. Additionally or alternatively, the cellular therapy can be chimeric antigen receptor (CAR) T-cell therapy or T-cell antigen coupler (TAC) T-cell therapy.

In some embodiments, administration of the nucleic acid molecule to the individual results in the cancer showing an increased sensitivity to the at least one additional therapeutic compound. In some embodiments, the regulator of the MHC molecule is selected from the group consisting of: transactivator, a transcription factor, an acetyltransferase, a methyltransferase, an elongation factor, and any combination thereof. In some embodiments, the transactivator is selected from the group consisting of: class II, major histocompatibility complex, transactivator (CIITA) and NOD-like receptor family CARD domain containing 5 (NLRC5). In some embodiments, the transcription factor is selected from the group consisting of: a nuclear transcription factor Y (NF-Y), cAMP response element-binding protein (CREB), a regulatory factor X (RFX), an interferon regulatory factor (IRF), a signal transducer and activator of transcription (STAT), a ubiquitous transcription factor (USF), and nuclear factor kappa-light-chain-enhancer of activated B cells (NF-κB). In some embodiments, the NF-Y is selected from the group consisting of: NF-Ya, NF-Yb, and NF-Yc. In some embodiments, the RFX is selected from the group consisting of RFXANK/RFXB, RFX5, and RFXAP. In some embodiments, the IRF is selected form the group consisting of IRF-1, IRF-2, IRF-3, IRF-4, IRF-5, IRF-6, IRF-7, IRF-8, and IRF-9. In some embodiments, the STAT is selected from the group consisting of STAT-1, STAT-2, STAT-3, STAT-4, STAT-5, and STAT-6. In some embodiments, the USF is selected from the group consisting of: USF-1 and USF-2. In some embodiments, the acetyltransferase is selected from the group consisting of CREB-binding protein (CBP), p300, and p300/CBP-associated factor (pCAF). In some embodiments, the methyltransferase is Enhancer of Zeste Homolog 2 (EZH2), protein arginine N-methyltransferase 1 (PRMT1), and coactivator-associated arginine methyltransferase 1 (CARM1). In some embodiments, the elongation factor is positive transcriptional elongation factor (pTEFb). In some embodiments, the nucleic acid molecule is DNA or RNA. In some embodiments, the nucleic acid is a plasmid. In some embodiments, the nucleic acid is a viral vector. In some embodiments, the viral vector is an alphavirus, a retrovirus, an adenovirus, a herpes virus, poxvirus, lentivirus, oncolytic virus, reovirus, or an adeno associated virus (AAV). In some embodiments, the nucleic acid is formulated for targeted delivery to a tumor cell. In some embodiments, the nucleic acid is formulated in a liposome. In some embodiments, the liposome comprises the additional therapeutic compound, a polyethylene glycol (PEG), a cell-penetrating peptide, a ligand, an aptamer, an antibody, or a combination thereof. In some embodiments, the liposome is formulated for targeted delivery to a cancer cell.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A-1D illustrate transfection of HLA-DR alleles in an RKO colonic carcinoma cell line. FIG. 1A shows no surface expression of any HLA receptor in parental RKO. FIG. 1B shows that in RKO transfected with HLADR A alone, there is no detected HLA-DR expression on the cell surface. However, intracellular expression for the Myc-DKK tag (data not shown) indicated successful transfection. FIG. 1C shows no HLA-DR surface expression in an RKO cell line transfected with HLADR B1 alone. However, GFP expression indicated successful transfection. FIG. 1D shows high and medium GFP expression with surface expression of both alpha and beta chains in HLA-DR A and B co-transfected cells.

FIGS. 2A-2C illustrate transfection of HLA-DR alleles in RKO colonic carcinoma and SKOV3 cell lines. FIG. 2A is a flow cytometry analysis of parental RKO cells. FIG. 2B is a flow cytometry analysis of GFP HLA-DRAB1*15 RKO cells. FIG. 2C shows punctate GFP in co-transfected RKO cells v. green fluorescent cytoplasm when only HLA-DR B was transfected.

FIGS. 3A-3D illustrate fluorescent pictures of stably co-transfected RKO and SKOV3 cells listed as: RKO HLA-DR AB1 (FIG. 3A); SKOV3 HLA-DR AB1 (FIG. 3B); RKO HLA-DR AB3 (FIG. 3C); and SKOV3 HLA-DR AB3 (FIG. 3D).

FIG. 4A illustrates a vector structure of HLA-DR B3.

FIG. 4B illustrates a vector structure of HLA-DR B4.

FIG. 4C illustrates a vector structure of HLA-DR B5.

FIG. 4D illustrates a vector structure of HLA-DR alpha a.

FIG. 4E illustrates a vector structure of HLA-DR B1*15.

FIG. 5A illustrates two representative dendritic cells prepared from two different donors expressing high levels of HLA-DR and PD-L1.

FIG. 5B illustrates primary T-cells prepared for the mixed lymphocute reaction (MLR) assays from two different donors genotyped as HLA-DR1.

FIG. 5C illustrate RKO cells expressing high levels of PD-L1.

FIGS. 6A-6F illustrate T cell proliferation when cultured with HLA-DR transfected RKO cells together with anti-PD-1 antibodies.

FIG. 6G illustrates that T cells were not proliferated when cultured with RKO parental cells.

FIG. 6H illustrates that T cells were not proliferated without any treatment.

FIG. 7A illustrates T cell proliferation when cultured with parental RKO cells together with anti-PD-1 antibodies.

FIG. 7B illustrates T cell proliferation when cultured with HLA-DR transfected RKO cells together with anti-PD-1 antibodies.

FIG. 7C illustrates T cell proliferation when cultured with HLA-DR transfected RKO cells.

FIG. 7D illustrates T cell proliferation when cultured with HLA-DR transfected RKO cells together with anti-PD-1 antibodies.

FIG. 8A-8C illustrate HLA-DR transfected RKO cells increased T cell proliferations and inflammatory cytokine secretion.

### DETAILED DESCRIPTION OF THE DISCLOSURE

Disclosed herein are immunotherapeutic compositions and methods of using the same to treat or prevent a condition such as cancer. An immunotherapeutic composition herein can comprise a nucleic acid molecule encoding an MHC component or a functional fragment thereof or a regulator of the nucleic acid molecule encoding the MHC component or functional fragment thereof. Further disclosed herein are immunotherapeutic compositions comprising a an MHC component polypeptide or a functional fragment thereof or a regulator of the nucleic acid molecule encoding the MHC component or functional fragment thereof.

### MHC Components

As used herein, “MHC component” or “MHC molecule” refers to a nucleic acid encoding an MHC gene, a polypeptide encoded by an MHC gene, a gene or gene product associated with an MHC, or a regulator of an MHC or a regulator of nucleic acids encoding an MHC component, or a functional fragment thereof. Thus, unless a sentence is limiting, the term MHC molecule should encompass both the nucleic acid sequences encoding an MHC protein as well as the proteins. Moreover, functional fragments refer to those fragments of the proteins and nucleic acid molecules that result in substantially the same function as the full sequence. So, in some embodiments, a functional fragment is the extracellular portion of a molecule described herein or the nucleic acid sequences encoding the extracellular portion of the protein. In other instances, a function fragment comprises both the extracellular domain and the transmembrane domain of a molecule (or nucleic acids encoding the same).

The MHC components herein can be mammalian MHC components, or more specifically a human MHC component, which can alternatively be referred to as a human leukocyte antigen (HLA). For example, HLA genes that are MHC components include HLA-A, HLA-B, HLA-C, HLA-E, HLA-F, HLA-G, HLA-H, HLA-J, HLA-K, HLA-N, HLA-P, HLA-S, HLA-T, HLA-U, HLA-V, HLA-W, HLA-X, HLA-Y, HLA-Z, HLA-DRA, HLA-DRB1, HLA-DRB2, HLA-DRB3, HLA-DRB4, HLA-DRB5, HLA-DRB6, HLA-DRB7, HLA-DRB8, HLA-DRB9, HLA-DQAT, HLA-DQB1, HLA-DQA2, HLA-DQB2, HLA-DQB3, HLA-DOA, HLA-DOB, HLA-DMA, HLA-DMB HLA-DPA1, HLA-DPB1, HLA-DPA2, HLA-DPB2, and HLA-DPA3. A gene or gene product associated with the MHC component can be β2 microglobulin (B2M). MHC component can be used to describe an entire MHC molecule or a portion or functional fragment thereof. An MHC molecule herein can be a MHC class I molecule, a non-classical MHC molecule, or a MHC class II molecule, or a homolog or functional fragment of any of the above.

Class I MHC molecules can present peptides derived from cytosolic proteins to cytotoxic T-cells to trigger an immune response. Class I MHC molecules can also present exogenous peptides through cross-presentation. The class I MHC molecule can comprise two domains: a heavy (α) chain and a light chain (β₂ microglobulin), wherein the heavy chain and the light chain are linked non-covalently. The heavy (α) chain can further comprise three extracellular domains: an α1 domain, an α2 domain, and an α3 domain, with the α2 domain and the α3 domain forming the groove to which the peptide that the class I MHC molecule presents is bound. Non-classical MHC I molecules of the disclosure can be recognized by natural killer (NK) cells and CD8⁺ T cells. HLA-E, HLA-F, and HLA-G are non-classical MHC I molecules encoded in the MHC I locus with low levels of heterogeneity compared to classical MHC I molecules. HLA-E molecule expression is IFN-γ-inducible and HLA-G expression can be induced by interferon-inducible transcription factors, such as IRF-1 and other stimuli.

The MHC components herein can be a class I MHC component or a functional fragment thereof. Examples of functional fragments include any of the above domains but not the entire MHC gene. For example, in one instance, an MHC component comprises the heavy (α) chain without a light chain (β₂ microglobulin). In other instances, an MHC component comprises a light chain (β₂ microglobulin) without the heavy (α) chain. In other instances, a class I MHC component can comprise a heavy (α) chain, a light chain (β₂ microglobulin), or a combination thereof. In some instances, an MHC component includes one or two of: an α1 domain, an α2 domain, and an α3 domain, but not all three domains.

A class I MHC component can be a human HLA-A gene, an HLA-B gene, an HLA-C gene or a polypeptide product thereof, or a homolog thereof, or functional fragment thereof. The class I MHC component can be a molecule encoded by any suitable HLA-A allele from a human genome. The class I MHC component can be a molecule encoded by any suitable HLA-B allele from a human genome. The class I MHC component can be a molecule encoded by any suitable HLA-C allele from a human genome. The class I MHC component can be a molecule encoded by any suitable β₂ microglobulin allele from a human genome. In some instances, the class I MHC component is a fragment of a class I MHC component. For example, a class I MHC component can be an exon or specific domain of a class I MHC component, such as the α2 domain and the α3 domain of the heavy chain. In some instances, the class I MHC component is a polypeptide encoded by a class I MHC gene. Thus, the present disclosure contemplates both the MHV and HLA polypeptide products and fragments (domains) described herein as well as nucleic acid molecules encoding the same.

The heavy chain of a class I MHC component can be functionally variable, wherein a plurality of different gene products can be produced by a single gene. The functionally variable products of a class I MHC gene can be referred to as a class I MHC serotypes. There can be at least 25 serotypes of HLA-A, at least 50 serotypes of HLA-B, and at least 12 serotypes of HLA-C. The class I MHC component can be any suitable class I MHC serotype. The class I MHC serotype can be HLA-A2, HLA-A3, or HLA-B8. The alleles representing these different serotypes can be selected from Table 3 attached herein. In some embodiments, a composition herein comprises nucleic acids encoding one or more, 2 or more, 3 or more, 4 or more, 5 or more, or 6 or more, 7 or more, 8 or more, 9 or more, or 10 or more different MHC components, HLA alleles, or HLA alleles described in Table 3, or functional fragments thereof.

A nucleic acid encoding a class I MHC component can comprise a nucleic acid encoding class I MHC component polypeptide. In one example, a nucleic acid encoding a class I MHC component comprises a nucleic acid that encodes an allele of HLA-A2, HLA-A3, or HLA-B8.

In some instances, the nucleic acid sequence encoding an MHC component is identical to a naturally occurring class I MHC nucleic acid sequence. In other instances, the nucleic acid sequence encoding an MHC component has been codon optimized or engineered for more efficient transfection or expression in a target cell. For example, in one instance, all intronic sequences are removed. In some instances, the nucleic acid molecule encoding an MHC component is non-naturally occurring, but the MHC component encoded by it has an amino acid sequence that is naturally occurring. This is true for all of the MHC components described herein. In some instances, the nucleic acid sequence is different from a naturally occurring class I MHC nucleic acid sequence but encodes a polypeptide identical to a class I MHC polypeptide owing to codon degeneracy. For example, a class I MHC nucleic acid sequence can be a codon optimized class I MHC nucleic acid sequence. In some instances, the nucleic acid encoding the class I MHC component comprises a nucleic acid optimized to improve expression of the class I MHC component. In some instances, the nucleic acid sequence encoding the class I MHC component is different from a naturally occurring class I MHC nucleic acid sequence but encodes a polypeptide identical to a class I MHC polypeptide and shows increased expression relative to the expression of a naturally occurring class I MHC nucleic acid sequence.

Further, the MHC component can be a non-classical MHC I component or a fragment thereof. Non-classical MHC-I molecules are usually nonpolymorphic and tend to show a more restricted pattern of expression than their MHC class I counterparts. The non-classical MHC I component can be a heavy (α) chain, a light chain (β₂ microglobulin), or a combination thereof. The non-classical MHC component can be an HLA-E gene, an HLA-G gene, an HLA-F gene or a polypeptide product thereof. The non-classical MHC component can be a molecule encoded by any suitable HLA-E allele from a human genome. The non-classical MHC component can be a molecule encoded by any suitable HLA-G allele from a human genome. The non-classical MHC component can be a molecule encoded by any suitable HLA-F allele from a human genome. The non-classical MHC component can be a molecule encoded by any suitable β₂ microglobulin allele from a human genome. In some instances, the non-classical MHC component is a functional fragment of a non-classical MHC component. For example, the non-classical MHC component can be an exon or specific domain of a non-classical MHC component, such as the α2 domain and the 3 domain of the heavy chain. In some instances, the class I MHC component is a polypeptide encoded by a non-classical MHC gene. Different alleles representing HLA-E, HLA-G, and HLA-F can be selected from Table 3.

A nucleic acid encoding a non-classical MHC I component can comprise a nucleic acid encoding a non-classical MHC I component. In some instances, the nucleic acid sequence is identical to a naturally occurring non-classical MHC I nucleic acid sequence. In some instances, the nucleic acid sequence is different from a naturally occurring non-classical MHC I nucleic acid sequence but encodes a polypeptide identical to a non-classical MHC I polypeptide owing to codon degeneracy. For example, a non-classical MHC I nucleic acid sequence can be a codon optimized non-classical MHC I nucleic acid sequence. In some instances, the nucleic acid encoding the non-classical MHC I component comprises a nucleic acid optimized to improve expression of the non-classical MHC I component. In some instances, the nucleic acid sequence encoding the non-classical MHC I component is different from a naturally occurring non-classical MHC I nucleic acid sequence but encodes a polypeptide identical to a non-classical MHC I polypeptide and shows increased expression relative to the expression of a naturally occurring non-classical MHC I nucleic acid sequence.

Class II MHC molecules can present peptides derived from extracellular proteins. These class II molecules can usually be found on antigen-presenting cells (APC), such as dendritic cells, macrophages, and B cells, although their expression can be induced in non-antigen-presenting cells such as tumor cells. A class II MHC molecule can comprise an alpha (α) chain and a beta (β) chain. The alpha chain can comprise an α1 domain and an α2 domain, while the beta chain can comprise a B1 domain and a B2 domain, with the α1 domain and the B1 domain forming the groove to which the peptide the class II MHC molecule presents is bound. In some instances, an MHC component comprises less than all of the domains of a Class II MHC molecule.

The MHC component can be a class II MHC component or a fragment thereof. The class II MHC component can be an alpha (α) chain, a beta (β) chain, or a combination thereof. The class II MHC component can be an HLA-DM gene, HLA-DO gene, an HLA-DP, an HLA-DQ gene, an HLA-DR gene, or a polypeptide product thereof. The alpha chains and beta chains for each of the HLA-DM, HLA-DO, HLA-DP, and HLA-DQ are described in Table 1. The class II MHC component can be a molecule encoded by any suitable HLA-DM, HLA-DO, HLA-DP, or HLA-DQ allele from a human genome. In some instances, the class II MHC component is a fragment of a class II MHC component. For example, the class II MHC component can be an exon or specific domain of a class II MHC component, such as the α1 domain of the alpha chain and the 31 domain of the beta chain. In some instances, the class II MHC component is a polypeptide encoded by a class II MHC gene in Table 1. In some instances, the class II MHC component is a polypeptide encoded by HLA-DR4 or HLA-DR15.

TABLE 1
Genes encoding alpha and beta chains of class II MHC molecules

| Class II MHC | | |
|---|---|---|
| molecule | Alpha chain | Beta chain |
| HLA-DM | HLA-DMA | HLA-DMB |
| HLA-DO | HLA-DOA | HLA-DOB |
| HLA-DP | HLA-DPA1, HLA-DPA2, | HLA-DPB1, HLA-DPB2 |
| | HLA-DPA3 | |
| HLA-DQ | HLA-DQA1, HLA-DQA2 | HLA-DQB1, HLA-DQB2, |
| | | HLA-DQB3 |
| HLA-DR | HLA-DRA | HLA-DRB1, HLA-DRB2, |
| | | HLA-DRB3, HLA-DRB4, |
| | | HLA-DRB5, HLA-DRB6, |
| | | HLA-DRB7, HLA-DRB8, |
| | | HLA-DRB9 |

A class II MHC component can be class II MHC molecule such as HLA-DM, HLA-DO, HLA-DP, HLA-DQ, or HLA-DR. Each of these class II MHC molecules can comprise an alpha chain and a beta chain encoded by a gene in Table 1. The alpha chain and beta chain genes in Table 1 can be functionally variable, wherein a plurality of different gene products can be produced by a single gene. In one example, different gene products can be produced by a single gene through alternative splicing of exons. The functionally variable products of an alpha chain and beta chain as shown in Table 1 can be referred to as a class II MHC serotypes. There can be at least 21 serotypes of HLA-DR, and at least 8 serotypes of HLA-DQ. The class II MHC component can be any suitable class II MHC serotype. The class II MHC component can be HLA-DR4 or HLA-DR15. The alleles representing these different serotypes can be selected from Table 3 attached herein.

A nucleic acid encoding a class II MHC component can comprise a nucleic acid encoding a class II MHC component. In some instances, the nucleic acid sequence is identical to a naturally occurring class II MHC nucleic acid sequence. In some instances, the nucleic acid sequence is different from a naturally occurring class II MHC nucleic acid sequence but encodes a polypeptide identical to a class II MHC polypeptide owing to codon degeneracy. For example, a class II MHC nucleic acid sequence can be a codon optimized class II MHC nucleic acid sequence. In some instances, the nucleic acid sequence encoding the class II MHC component is different from a naturally occurring class II MHC nucleic acid sequence but encodes a polypeptide identical to a class II MHC polypeptide and shows increased expression relative to the expression of a naturally occurring class II MHC nucleic acid sequence. In some instances, the nucleic acid encoding the class II MHC component comprises a nucleic acid optimized to improve expression of the class II MHC component. In some instances, the nucleic acid sequence encoding the class II MHC component is different from a naturally occurring class II MHC nucleic acid sequence but encodes a polypeptide identical to a class II MHC polypeptide and shows increased expression relative to the expression of a naturally occurring class II MHC nucleic acid sequence.

Disclosed herein, in certain embodiments is a non-naturally occurring MHC component or a fragment thereof. In some instances, the non-naturally occurring MHC component is a homolog of any of a class I MHC component or class II MHC component. A homolog is a non-naturally occurring sequence that has high sequence similarity or sequence identity to a naturally occurring sequence.

In general, “sequence similarity,” “sequence identity,” or “sequence homology,” which can be used interchangeably, refer to an exact nucleotide-to-nucleotide or amino acid-to-amino acid correspondence of two polynucleotides or polypeptide sequences, respectively. Typically, techniques for determining sequence identity include determining the nucleotide sequence of a polynucleotide and/or determining the amino acid sequence encoded thereby, and comparing these sequences to a second nucleotide or amino acid sequence. Two or more sequences (polynucleotide or amino acid) can be compared by determining their “percent identity”, also referred to as “percent homology”. The percent identity to a reference sequence (e.g., nucleic acid or amino acid sequences), which may be a sequence within a longer molecule (e.g., polynucleotide or polypeptide), may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin and Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the sequences being compared. Default parameters are provided to optimize searches with short query sequences, for example, with the blastp program. The program also allows use of an SEG filter to mask-off segments of the query sequences as determined by the SEG program of Wootton and Federhen, Computers and Chemistry 17: 149-163 (1993). A high sequence identity between a disclosed sequence and a claimed sequence contemplates at least 80%, at least 85%, at least 90%, at least 95%, at least 98%, or at least 99%. In some cases, reference to percent sequence identity refers to sequence identity as measured using BLAST (Basic Local Alignment Search Tool). As used herein, percent sequence identity or homology can be determined by any one or more of the conventional methods. Methods for analyzing sequence homology include, but are not limited to, pairwise sequence alignment, which is used to identify regions of similarity that may indicate functional, structural and/or evolutionary relationships between two biological sequences (protein or nucleic acid); and multiple sequence alignment (MSA), which is an alignment of three or more biological sequences of similar length. Various software and analytic tools are available for determining sequence homology based on global alignment, local alignment, or genomic alignment. Examples include, but are not limited to, EMBOSS Needle provides an optimal global alignment of two sequences using the Needleman-Wunsch algorithm; EMBOSS Stretcher uses a modified version of the Needleman-Wunsch algorithm that allows larger sequences to be globally aligned; EMBOSS Water uses the Smith-Waterman algorithm to calculate local alignment of two sequences; EMBOSS Matcher provides local similarities between two sequences using a rigorous algorithm based on the LALIGN application; LALIGN identifies internal duplications by calculating non-intersecting local alignments of protein or DNA sequences; Wise2DBA (DNA Block Aligner) aligns two sequences based on the assumption that the sequences share a number of colinear blocks of conservation separated by potentially large and varied lengths of DNA in the two sequences; GeneWise compares a protein sequence to a genomic DNA sequence, allowing for introns and frameshifting errors; PromoterWise compares two DNA sequences allowing for inversions and translocations, ideal for promoters; BLAST provides local search with fast k-tuple heuristic; FASTA provides local search with fast k-tuple heuristic, faster but less sensitive than BLAST; and ClustalW provides local or global progressive alignment. In some cases, ClustalW can be used for multiple sequence alignment. In some cases, Smith-Waterman and/or BLAST can be used to find homologous sequences by searching and comparing a query sequence with sequences in a database. In some cases, Smith-Waterman algorithm is preferably used to determine sequence identity within a domain or for local sequence alignment instead of comparing full-length or entire sequences, as the Smith-Waterman algorithm compares segments of all possible lengths and optimizes the similarity measure. In some cases, the Needleman-Wunsch algorithm is preferably used for aligning entire protein or nucleotide sequences to determine global or overall sequence identity. EMBOSS Needle and Stretcher tools use the Needleman-Wunsch algorithm for global alignment. EMBOSS Water tool uses the Smith-Waterman algorithm for local alignment. In various embodiments disclosed herein, overall or local sequence identity is determined preferably using BLAST.

The non-naturally occurring MHC component can show expression in a cell that does not normally express a corresponding naturally occurring MHC component. The non-naturally occurring MHC component can show enhanced expression by a cell relative to a naturally occurring MHC component. Expression of the non-naturally occurring MHC component by the cell can result in enhanced recognition by a T-cell relative to a naturally occurring MHC component. Expression of the non-naturally occurring MHC component can result in increased apoptosis of the cell expressing the non-naturally occurring MHC component. The cell can be a tumor cell.

A nucleic acid encoding a non-naturally occurring MHC component can comprise at least one variant compared to a nucleic acid molecule encoding a naturally occurring MHC component. The variant can be a mutation, an insertion, a deletion, or a duplication. The mutation can result in a substitution, which can further encode a synonymous or non-synonymous mutation, a frameshift mutation, or a nonsense mutation. In some instances, the mutation is in a protein coding portion of a gene encoding the non-naturally occurring MHC component. In some instances, the mutation is in a promoter region of the gene encoding the non-naturally occurring MHC component.

The nucleic acid molecule of the non-naturally occurring MHC component can be at least 20%, at least 30%, at least 40%, at least 50%, at least 60%, at least 70% at least 80%, at least 85%, at least 90%, at least 95%, or at least 99% similar to the nucleic acid sequence encoding a corresponding naturally occurring MHC component. In some instances, the nucleic acid molecule is at least 20% similar to the nucleic acid sequence encoding the naturally occurring MHC component. In some instances, the nucleic acid molecule is at least 80% similar to the nucleic acid sequence encoding the naturally occurring MHC component. In some instances, the nucleic acid molecule is at least 95% similar to the nucleic acid sequence encoding the naturally occurring MHC component.

The polypeptide of the non-naturally occurring MHC component can be at least 80%, at least 85%, at least 90%, at least 95%, or at least 99% similar to the polypeptide of the naturally occurring MHC component. In some instances, the polypeptide is at least 80% similar to the polypeptide of the naturally occurring MHC component. In some instances, the polypeptide is at least 95% similar to the polypeptide of the naturally occurring MHC component.

Regulators of MHC molecules can be regulators of class I MHC molecules or class II MHC molecules. The regulator can regulate transcription of a nucleic acid encoding the MHC molecule. Regulation of the transcription of the nucleic acid encoding the MHC molecule can comprise an increase in the level of transcription of the MHC molecule. Regulation of the transcription of the nucleic acid encoding the MHC molecule can comprise a decrease in the level of transcription of the MHC molecule. The regulator can be a transactivator, a transcription factor, an acetyltransferase, a methyltransferase, an elongation factor, or any combination thereof.

The transactivator can be class II, major histocompatibility complex, transactivator (CIITA) or NOD-like receptor family CARD domain containing 5 (NLRC5). In some instances, CIITA is a transactivator for class II MHC molecules. In some instances NLRC5 is a transactivator for class I MHC molecules.

The transcription factor can be a nuclear transcription factor Y (NF-Y), cAMP response element-binding protein (CREB), a regulatory factor X (RFX), an interferon regulatory factor (IRF), a signal transducer and activator of transcription (STAT), a ubiquitous transcription factor (USF), or nuclear factor kappa-light-chain-enhancer of activated B cells (NF-κB). The NF-Y can be NF-Ya, NF-Yb, or NF-Yc. The RFX can be RFXANK/RFXB, RFX5, or RFXAP. The IRF can be IRF-1, IRF-2, IRF-3, IRF-4, IRF-5, IRF-6, IRF-7, IRF-8, or IRF-9. The STAT can be STAT-1, STAT-2, STAT-3, STAT-4, STAT-5, or STAT-6. The USF can be USF-1 or USF-2.

The acetyltransferase can be a histone acetyltransferase (HAT). The HAT can be a CREB-binding protein (CBP), p300, or p300/CBP-associated factor (pCAF). In some embodiments, the regulator is a histone deacetylase inhibitor (DAI).

The methyltransferase can be a histone methyltransferase (HMTase), a DNA/RNA methyltransferase, or an arginine methyltransferase. The HTMase can be Enhancer of Zeste Homolog 2 (EZH2). The arginine methyltransferase can be protein arginine N-methyltransferase 1 (PRMT1) or coactivator-associated arginine methyltransferase 1 (CARM1). In one example, decreased expression of EZH2 can increase expression of CIITA.

The elongation factor can be a positive transcriptional elongation factor (pTEFb).

In some embodiments, regulators of MHC molecules are upregulated by an additional factor. The additional factor upregulating a regulator of an MHC molecule can be IFN-γ, lipopolysaccharide (LPS), or IL-4. In other embodiments, regulators of MHC molecules are downregulated by an additional factor. The additional factor downregulating a regulator of an MHC molecule can be IFN-β, IL-10, nitric oxide (NO), or TGFβ. The regulator of an MHC molecule upregulated or downregulated by an additional factor can be CIITA or NLRC5.

Regulators of MHC molecules can be a ligand of a costimulatory molecule. The costimulatory molecule can be a molecule required for T-cell activation. A costimulatory molecule can be CD40. The regulator of an MHC molecule can be a ligand of CD40.

### Immunotherapeutic Compositions

Disclosed herein, in certain embodiments, are immunotherapeutic compositions comprising a nucleic acid molecule encoding an MHC component or a fragment thereof. In certain embodiments, the immunotherapeutic compositions comprise a polypeptide of an MHC component or a fragment thereof. Further disclosed herein, in certain embodiments, are immunotherapeutic compositions comprising a nucleic acid molecule encoding a regulator of an MHC component or a fragment thereof or a polypeptide of a regulator of an MHC component or a fragment thereof. The nucleic acid molecule can be DNA or RNA. Any of the MHC components herein can be used as immunotherapeutic compositions.

The immunotherapeutic composition can comprise a nucleic acid molecule encoding a class I MHC component, such as a class I MHC heavy (α) chain. The nucleic acid molecule can further encode a second class I MHC component, such as a class I MHC light chain (β₂ microglobulin). For example, the immunotherapeutic composition can comprise a nucleic acid molecule encoding a class I MHC heavy (α) chain and a class I MHC light chain (β₂ microglobulin). In some instances, the immunotherapeutic composition further comprises a second nucleic acid molecule encoding a second class I MHC component. For example, the immunotherapeutic composition can comprise a first nucleic acid molecule encoding a class I MHC heavy (α) chain and a second nucleic acid molecule encoding a class I MHC light chain (β₂ microglobulin).

The immunotherapeutic composition can comprise a nucleic acid molecule encoding a class II MHC component, such as a class II MHC alpha (α) chain. The nucleic acid molecule can further encode a second class II MHC component, such as a class II MHC beta (β) chain. For example, the immunotherapeutic composition can comprise a nucleic acid molecule encoding a class II MHC alpha (α) chain and a class II MHC beta (β) chain. In some instances, the immunotherapeutic composition further comprises a second nucleic acid molecule encoding a second class II MHC component. For example, the immunotherapeutic composition can comprise a first nucleic acid molecule encoding a class II MHC alpha (α) chain and a second nucleic acid molecule encoding a class II MHC beta (β) chain.

The immunotherapeutic composition can comprise a nucleic acid encoding a regulator of an MHC component or a fragment thereof. The immunotherapeutic composition can comprise a polypeptide of a regulator of an MHC component or a fragment thereof. The regulator can be a transactivator, a transcription factor, an acetyltransferase, a methyltransferase, an elongation factor, or any combination thereof as previously described herein. The immunotherapeutic composition can comprise an additional factor regulating a regulator of an MHC component or fragment thereof. The additional factor regulating the regulator of the MHC component can be IFN-γ, lipopolysaccharide (LPS), IL-4, IFN-β, IL-10, nitric oxide (NO), or TGFβ. The additional factor can be administered as a polypeptide or as a small molecule (e.g. NO).

The additional factor can be administered simultaneous with the nucleic acid encoding the regulator of the MHC component or fragment thereof. The additional factor can be administered sequentially following administration of the nucleic acid encoding the regulator of the MHC component or fragment thereof. The nucleic acid encoding the regulator of the MHC component or fragment thereof can be administered sequentially following administration of the additional factor.

The immunotherapeutic composition can comprise a ligand of a costimulatory molecule. The costimulatory molecule can be CD40.

The immunotherapeutic composition can comprise a nucleic acid encoding a deactivated CRISPR-associated nuclease fused to a TET enzyme. The nucleic acid encoding a deactivate CRIPSR-associated nuclease fused to a TET enzyme can further encode at least one guide RNA (gRNA). The immunotherapeutic composition comprising a nucleic acid encoding a deactivate CRIPSR-associated nuclease fused to a TET enzyme can further comprise a second nucleic acid encoding the gRNA. The gRNA can comprise a region complementary to a transcription factor, a regulator of an MHC component, or a promoter of an MHC gene. The deactivated CRISPR-associated nuclease can be a deactivated Cas9 (dCas9) or a deactivated Cpf1 (dCfpl). The TET enzyme can be TET1, TET2, TET3, or a catalytic domain thereof. In some instance, the TET enzyme is a TET1 enzyme or a catalytic domain of the TET1 enzyme. Administration of an immunotherapeutic composition comprising a nucleic acid encoding a deactivated CRISPR-associated nuclease fused to a TET enzyme can be used to demethylate a promoter, a regulator of an MHC component, or a transcription factor associated with an MHC gene. Demethylating a promoter, a regulator of an MHC component, or transcription factor associated with an MHC gene can result in increased expression of the MHC gene.

The immunotherapeutic composition can further comprise at least a second nucleic acid encoding a second deactivated CRISPR-associated nuclease fused to a TET enzyme. The second nucleic acid can further encode at least one second guide RNA. In some instances, the immunotherapeutic composition comprises a plurality of nucleic acids encoding a deactivated CRISPR-associated nuclease fused to a TET enzyme and a plurality of guide RNAs. In some instances, the immunotherapeutic composition comprises a single nucleic acid encoding a deactivated CRISPR-associated nuclease fused to a TET enzyme and a plurality nucleic acids encoding a plurality of guide RNAs. In some instances, a gRNA is designed to target a single methylated CpG site. In other instances, the gRNA is designed to target at least two methylated CpG sites.

The immunotherapeutic composition can be formulated as an aqueous solution. The immunotherapeutic composition can be formulated as a powder, for example a dry powder nucleic acid composition comprising a lipid-DNA complex. The powder formulation can further be suspended in an aqueous solution. The immunotherapeutic composition can be lyophilized, sterilized, or a combination thereof.

The immunotherapeutic composition can further comprise at least pharmaceutically acceptable excipient. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Any suitable pharmaceutically acceptable excipient can be used. An excipient can be a carrier, a diluent, a detergent, a buffer, a salt, a peptide, a surfactant, an oligosaccharide, an amino acid, a carbohydrate, or an adjuvant. In some instances, a hydrophilic excipient is used, for example a dry powder immunotherapeutic composition comprising nucleic acid dispersed within a hydrophilic excipient. Examples of excipients include, but are not limited to, human serum albumin, collagen, gelatin, hyaluronic acid, glucose, lactose, sucrose, xylose, ribose, trehalose, mannitol, raffinose, stachyose, dextran, maltodextrin, cylcodextrin, cellulose, methylcellulose, glycine, alanine, glutamate, ascorbic acid, ascorbate salts, citric acid, citrate salts, NaCl, NaHCO₃, NH₄HCO₃, MgSO₄, and Na₂SO₄.

In some instances, excipients are used to stabilize the immunological composition. The excipient can be salts dissolved in buffered solutions (which also can provide pH control or maintenance), including, but not limited to a phosphate buffered saline solution. In some instances, the excipient increases bulk of the immunological composition. The excipient can increase or decrease the absorption of the immunological composition by the individual.

The compositions herein can be formulated for oral delivery, or delivery that is intravenous, intramuscular, subcutaneous, subdermal, subcutaneous, sublingual, as well as other routes.

Solid dosage forms suitable for oral administration in accordance with the present teachings include but are not limited to capsules, tablets, pills, powders, and granules. In such solid dosage forms, the active compound is mixed with at least one inert, pharmaceutically acceptable excipient or carrier such as sodium citrate or dicalcium phosphate and/or (a) fillers or extenders such as starches, lactose, sucrose, glucose, mannitol, and silicic acid; (b) binders such as, for example, carboxymethylcellulose, alginates, gelatin, polyvinylpyrrolidinone, sucrose, and acacia; (c) humectants such as glycerol; (d) disintegrating agents such as agar-agar, calcium carbonate, potato or tapioca starch, alginic acid, certain silicates, and sodium carbonate; (e) solution retarding agents such as paraffin; (f) absorption accelerators such as quaternary ammonium compounds; (g) wetting agents such as, for example, acetyl alcohol and glycerol monostearate; (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

The active compounds may also be in micro-encapsulated form with one or more excipients as noted above. Encapsulation can include the use of liposomes, exosomes, lipid nanoparticles, or a biomaterial.

Liquid dosage forms for oral administration include but are not limited to pharmaceutically acceptable emulsions, microemulsions, solutions, suspensions, syrups and elixirs.

Injectable preparations (e.g., sterile injectable aqueous or oleaginous suspensions) may be formulated according to the known art using suitable dispersing or wetting agents and suspending agents.

Any of the formulations or compositions herein are preferably designed to specifically target cancer cells. For example, in some instances, the MHC component is formulated in an exosome that selectively targets cancer cells. Examples of such exosomes are described in Gomari et al., Onco Targets (2018) 11: 5753-5762 “Targeted cancer therapy using engineered exosome as a natural drug delivery vehicle.” In some instances, the MHC component or a vesicle encapsulating the same comprises an aptamer that selectively targets the MHC component or the vesicle encapsulating it to a cancer cell. Examples of aptamers that selectively target cancer cells are described in Cerchia et al, Trends Biotechnol. (2010) October 28(10): 517-25 “Targeting cancer cells with nucleic acid aptamers”. In another example, the MHC component or vesicle encapsulating it is coupled to a nano-material that selectively targets cancer cells, such as cancer stem cells. Examples of such nano-materials include those described in Qin et al. (2017) Front. Pharmacol. “Nanomaterials in targeting cancer stem cells for cancer therapy”. In another example, the MHC component or vesicle encapsulating it is coupled to an antibody that selectively targets cancer stem cells. This can form a drug-antibody conjugate. Or alternatively the antibody can be displayed on the surface of a vesicle that directs an encapsulated MHC component to the cancer cells. Examples of drug antibody conjugation is described in Thomas et al, (2016) Lancet Oncol., June 17(6), “Antibody-drug conjugates for cancer therapy” and Dan et al., (2018) Pharmaceutical (Basel) (2018) June; 11(2):32, “Antibody-drug conjugates for cancer therapy: chemistry to clinical implications.”

A nucleic acid encoding an MHC component, a nucleic acid encoding a regulator of the MHC component, or a nucleic acid encoding a deactivated CRISPR-associated nuclease fused to a TET enzyme can be delivered to the cell via a vector. The nucleic acid can be RNA or DNA. The cell can be a tumor cell. The vector can be a viral vector or a non-viral vector. Non-viral vector delivery systems include DNA plasmids, RNA (e.g. a transcript of a vector described herein), naked nucleic acid, and nucleic acid complexed with a delivery vehicle, such as a lipid or a liposome.

A lipid can be a cationic lipid, an anionic lipid, or neutral lipid. The lipid can be a liposome, a small unilamellar vesicle (SUV), a lipidic envelope, a lipidoid, or a lipid nanoparticle (LNP). The lipid can be mixed with the nucleic acid to form a lipoplex (a nucleic acid-liposome complex). The lipid can be conjugated to the nucleic acid. The lipid can be a non-pH sensitive lipid or a pH-sensitive lipid. The lipid can further comprise a polythethylene glycol (PEG).

The cationic lipid can be a monovalent cationic lipid, such as N-[1-(2,3-dioleyloxy)propyl]-N,N,N-trimethylammonium chloride (DOTMA), [1,2-bis(oleoyloxy)-3-(trimethylammonio)propane](DOTAP), or 3β[N—(N′, N′-dimethylaminoethane)-carbamoyl]cholesterol (DC-Chol). The cationic lipid can be a multivalent cationic lipid, such as Di-octadecyl-amido-glycyl-spermine (DOGS) or {2,3-dioleyloxy-N-[2(sperminecarboxamido)ethyl]-N,N-dimethyl-l-propanaminium trifluoroacetate}(DOSPA).

The anionic lipid can be a phospholipid or dioleoylphosphatidylglycerol (DOPG). Examples of phospholipids include, but are not limited to, phosphatidic acid, phosphatidylglycerol, or phosphatidylserine. In some instances, the anionic lipid further comprises a divalent cation, such as Ca²⁺, Mg²⁺, Mn2+, and Ba²⁺.

The cationic lipid or the anionic lipid can further comprise a neutral lipid. The neutral lipid can be dioleoylphosphatidyl ethanolamine (DOPE) or dioleoylphosphatidylcholine (DOPC). In some instances, the use of a helper lipid in combination with a charged lipid yields higher transfection efficiencies.

The liposome can further comprise a polymer, a lipid, a peptide, a magnetic nanoparticle (MNP), an additional compound, or a combination thereof. The polymer, lipid, or magnetic nanoparticle can be attached to the liposome or integrated into the liposomal membrane. The polymer can be a polyethylene glycol (PEG). The polymer can be N-[2-hydroxypropyl]methacrylamide (HPMA), poly(2-(dimethylamino)ethyl methacrylate) (pDMAEMA), or arginine-grafted bioreducible polymers (ABPs). The peptide can be a cell-penetrating peptide, a cell adhesion peptide, or a peptide which binds to a receptor on a cell. The cell can be a tumor cell. Any suitable cell-penetrating peptide can be used. Examples of cell-penetrating peptides include, but are not limited to a polylysine peptide and a polyarginine peptide. The cell adhesion peptide can be an arginylglycylaspartic acid (RGD) peptide. An additional compound can be a compound which binds to a receptor on a cell, such as folic acid.

The vector can be a viral vector. The viral vector can be a replication-competent viral vector or a replication-incompetent viral vector. The viral vector can be an oncolytic virus. Examples of viral vectors include, but are not limited to, an alphavirus, a retrovirus, an adenovirus, a herpes virus, poxvirus, lentivirus, oncolytic virus, reovirus, or an adeno associated virus (AAV). The alphavirus can be a Semliki Forest virus (SFV), a Sindbis virus (SIN), or a Venezuelan Equine Encephalitis (VEE). The pox virus can be a vaccinia virus. The herpes virus can be a herpes simplex virus (HSV) or an Epstein-barr virus (EBV). The adeno associated virus can be AAV1, AAV2, AAV3, AAV4, AAV5, AAV6, AAV7, or AAV8.

The viral vector can be a modified viral vector. The modified viral vector can show reduced immunogenicity, an increase in the persistence of the vector in the blood stream, or impaired uptake of the vector by macrophages and antigen presenting cells.

The modified viral vector can further comprise a polymer, a lipid, a peptide, a magnetic nanoparticle (MNP), an additional compound, or a combination thereof. The polymer, lipid, or magnetic nanoparticle can be attached to a capsid of the viral vector. The polymer can be a polyethylene glycol (PEG). The polymer can be N-[2-hydroxypropyl]methacrylamide (HPMA), poly(2-(dimethylamino)ethyl methacrylate) (pDMAEMA), or arginine-grafted bioreducible polymers (ABPs). The peptide can be a cell-penetrating peptide, a cell adhesion peptide, or a peptide which binds to a receptor on a cell. The cell can be a tumor cell. Any suitable cell-penetrating peptide can be used. Examples of cell-penetrating peptides include, but are not limited to a polylysine peptide and a polyarginine peptide. The cell adhesion peptide can be an arginylglycylaspartic acid (RGD) peptide. An additional compound can be a compound which binds to a receptor on a cell, such as folic acid.

The magnetic nanoparticle can be a superparamagnetic nanoparticle. In some instances, binding of an MNP can result a lower viral vector dose for optimal transgene delivery. In some instances, binding of an MNP improves transduction efficiency.

In some instances, the modified viral vector is a genetically modified vector. The genetically modified vector can have reduced immunogenicity, reduced genotoxicity, increased loading capacity, increased transgene expression, or a combination thereof. In some instances, the genetically modified viral vector is a pseudotyped viral vector. The pseudotyped viral vector can have at least one foreign viral envelope protein. The foreign viral envelope protein can be an envelope protein from a lyssavirus, an arenavirus, a hepadnavirus, a flavivirus, a paramyxovirus, a baculovirus, a filovirus, or an alphavirus. The foreign viral envelope protein can be the glycoprotein G of a vesicular stomatitis virus (VSV). In some instances, the foreign viral envelope protein is a genetically modified viral envelope protein. The genetically modified viral envelope protein can be a non-naturally occurring viral envelope protein.

In some instances, a capsid of the viral vector is conjugated with a bi-specific antibody. The bi-specific antibody can be targeted to bind to a cell of interest. The cell of interest can be a tumor cell.

Any of the compositions and immunotherapies herein can further comprise one or more therapeutic moieties. Such therapeutic moieties can include an immune checkpoint inhibitor, an immune checkpoint stimulator, a cancer vaccine, a small molecule therapy, a monoclonal antibody, a cytokine, a cellular therapy, or a combination thereof.

### METHOD OF USE

The compositions herein can be used to increase T cell activation and/or cytokine release. This can occur in vivo or in vitro. Such methods can further be used to treat conditions that evade the immune system, such as cancer for example. Thus described herein, in certain embodiments, are methods for activating the immune system and/or enhancing T cell activity and/or increasing cytokine mediated response in a subject. Such cytokine releases may be of interferon-gamma and TNF alpha. Also described herein, in certain embodiments, are methods of treating a cancer in an individual, comprising administering to the individual an immunotherapeutic composition comprising a nucleic acid molecule encoding an MHC component or polypeptide thereof. Further described herein, in certain embodiments, are methods of treating a cancer in an individual, comprising administering to the individual an immunotherapeutic composition comprising a nucleic acid molecule encoding a regulator of an MHC component, or a polypeptide thereof. Further described herein, in certain embodiments, are methods of treating a cancer in an individual, comprising administering to the individual an immunotherapeutic composition comprising a nucleic acid molecule encoding an nucleic acid encoding a deactivated CRISPR-associated nuclease fused to a TET enzyme. In some cases, methods of treating a cancer in an individual comprise administering to the individual an immunotherapeutic composition comprising at least one nucleic acid molecule encoding at least two of the following: an MHC component, a regulator of an MHC component, an additional factor regulating a regulator of an MHC molecule, and a deactivated CRISPR-associated nuclease fused to a TET enzyme. The at least one nucleic acid molecule can comprise 1, 2, 3, 4, 5, 6, 7, 8, 9, 10, or more than 10 nucleic acid molecules. Thus, a composition herein can include at least 1, 2, 3, 4, 5, 6, 7, 8, 9, or 10 different nucleic acid molecules either operably linked to one another or in separate plasmids, each of which includes a nucleic acid molecule encoding an MHC component.

The compositions herein can be used to treat cancer. The cancer can be solid tumor cancer, hematological cancer, acute myeloid leukemia, chronic lymphocytic leukemia, chronic myeloid leukemia, acute lymphoblastic leukemia, non-Hodgkin lymphoma, Hodgkin lymphoma, multiple myeloma, bladder cancer, pancreatic cancer, cervical cancer, endometrial cancer, lung cancer, bronchus cancer, liver cancer, ovarian cancer, colon and rectal cancer, stomach cancer, gastric cancer, gallbladder cancer, gastrointestinal stromal tumor cancer, thyroid cancer, head and neck cancer, oropharyngeal cancer, esophageal cancer, melanoma, non-melanoma skin cancer, Merkel cell carcinoma, virally induced cancer, neuroblastoma, breast cancer, prostate cancer, renal cancer, renal cell cancer, renal pelvis cancer, leukemia, lymphoma, sarcoma, glioma, brain tumor, and carcinoma. In some instances, the cancer is ovarian cancer, pancreatic cancer, or colon cancer. The cancer can be a cancer that does not express an MHC molecule. The cancer can be a cancer that shows reduced expression of the MHC molecule. The MHC molecule can be a class I MHC molecule or a class II MHC molecule. In some instances, the cancer is a cancer that does not respond to an immune checkpoint inhibitor therapy.

In some instances, the method further comprises diagnosing the cancer with no or reduced MHC molecule expression. Diagnosing the cancer with no or reduced MHC molecule expression can comprise: (a) obtaining a biological sample from the individual, (b) isolating cancerous cells from the biological sample; and (c) detecting whether MHC molecule expression in the isolated cancerous cells is reduced or eliminated relative to a control. The control can be a predetermined level, the level of MHC expression in a non-cancerous tissue of the individual, or a level of MHC molecule expression in a non-cancerous tissue of a different subject.

In some instances, the method further comprises determining the sequence of an MHC component of the individual. The sequence of the MHC component can include exons and introns of an MHC gene as well as a promoter, 5′UTR, and 3′UTR region thereof. The MHC component of the individual can be the sequence of the native or endogenous MHC component of the individual. Sequencing the MHC component of the individual can comprise Sanger or next generation sequencing (NGS). Sequencing the MHC component can further comprise an initial step of treating the nucleic acid of the individual with bisulfite prior to sequencing. Comparing a nucleic acid sequence to a bisulfite treated nucleic acid sequence can be used to identify methylated CpG sites. In some instances, sequencing the MHC component of the individual is informative for the desired sequence of the immunotherapeutic composition. For example, if a promoter of an MHC component from a cancerous cell is hypermethylated compared to the MHC component from a non-cancerous cell, an immunotherapeutic composition can be designed to demethylate at least one methylated CpG site of the promoter. In another example, sequencing the MHC component of the individual allows for a non-naturally MHC component to be designed which will be immunologically compatible with the individual.

The method can further comprising administering an additional therapeutic compound to the individual. The additional therapeutic compound can be a therapeutic agent which binds to an immune checkpoint gene or a ligand thereof, a cancer vaccine, a small molecule therapy, a monoclonal antibody, a cytokine, a cellular therapy, or a combination thereof. The therapeutic agent which binds to an immune checkpoint molecule or a ligand thereof can be an immune checkpoint inhibitor or an immune checkpoint agonist. Examples of immune checkpoint molecules include, but are not limited to, CD27, CD28, CD40, CD122, OX40, ICOS, A2AR, B7-H3, B7-H4, BTLA, CTLA-4, IDO, KIR, LAG3, PD-1, TIM-3, VISTA, 4-1BB, and GITR. Examples of immune checkpoint inhibitors include, but are not limited to, Ipilimumab, Tremelimumab, Nivolumab, Pembrolizumab, Atezolizumab, Avelumab, Durvaumab, and Lirilumab. The small molecule therapy can be a proteasome inhibitor, a tyrosine kinase inhibitor, a cyclin-dependent kinase inhibitor, or a polyADP-ribose polymerase (PARP) inhibitor. The cytokine can be INFα, INFβ, IFNγ, or TNF. The cellular therapy can be an adoptive T cell transfer (ACT) therapy. Additionally or alternatively, the cellular therapy can be chimeric antigen receptors (CARs) T cell therapy or T-cell antigen couplers (TACs) T cell therapy. TAC receptors operate through the native T-cell receptors (TCRs). Further, a TAC comprises (1) an antigen-binding domain, (2) a TCR recruitment domain, and (3) a co-receptor domain (hinge, transmembrane, and cytosolic regions).

The additional therapeutic compound can be administered simultaneous with administration of the immunotherapeutic compound, or can be administered before or after administration of the immunotherapeutic compound. In some instances, administration of the immunotherapeutic composition results in the cancer showing an increased sensitivity to the at least one additional therapeutic compound.

In some instances, a immunotherapeutic composition is delivered via a variety of routes. Exemplary delivery routes include oral (including buccal and sub-lingual), rectal, nasal, topical, transdermal patch, pulmonary, vaginal, suppository, or parenteral (including intramuscular, intraarterial, intrathecal, intradermal, intraperitoneal, subcutaneous and intravenous) administration or in a form suitable for administration by aerosolization, inhalation or insufflation. In some instances, the immunotherapeutic composition described herein is administered to muscle, or can be administered via intradermal or subcutaneous injections, or transdermally, such as by iontophoresis. In some cases, epidermal administration of the immunotherapeutic composition is employed.

The immunotherapeutic composition can be administered to a subject in need thereof, for example, one or more times (e.g., 1-10 times or more) daily, weekly, monthly, biannually, annually, or as medically necessary. Dosages may be provided in either a single or multiple dosage regimens. The timing between administrations can decrease as the medical condition improves or increase as the health of the patient declines.

The dosage of the pharmaceutical compositions of the disclosure depends on factors including the route of administration, the disease to be treated, and physical characteristics, e.g., age, weight, general health, of the subject. Typically, the amount of the pharmaceutical composition contained within a single dose can be an amount that effectively prevents, delays, or treats the disease without inducing significant toxicity. The effective amount for use in humans can be determined from animal models. For example, a dose for humans can be formulated to achieve circulating, liver, topical, and/or gastrointestinal concentrations that have been found to be effective in animals. Based on animal data, and other types of similar data, those skilled in the art can determine the effective amounts of a vaccine composition appropriate for humans. The dosage can be adapted by the physician in accordance with conventional factors such as the extent of the disease and different parameters of the subject.

The immunotherapeutic composition can be administered before, during, or after the onset of a symptom associated with a disease or condition (e.g., a cancer). In some instances, the immunotherapeutic composition is administered for treatment of a cancer. In some cases, the immunotherapeutic composition is administered for prevention, such as a prophylactic treatment of a cancer. In some cases, the immunotherapeutic composition is administered to illicit an immune response from a patient.

In some aspects, the immunotherapeutic composition and kit described herein are stored at between 2 °C and 8 °C. In some instances, the immunotherapeutic composition is not stored frozen. In some instances, the immunotherapeutic composition is stored in temperatures of such as at −20 °C or −80 °C. In some instances, the immunotherapeutic composition is stored away from sunlight.

### Kits

Disclosed herein, in certain embodiments, are kits and articles of manufacture for use with one or more methods described herein. The kit can comprise an immunotherapeutic composition described herein formulated in a compatible pharmaceutical excipient and placed in an appropriate container.

The kit can include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. A container can be formed from a variety of materials such as glass or plastic.

The kit can include an identifying description, a label, or a package insert. The label or package insert can list contents of kit or the immunological composition, instructions relating to its use in the methods described herein, or a combination thereof. The label can be on or associated with the container. The label can be on a container when letters, numbers, or other characters forming the label are attached, molded or etched into the container itself. The label can be associated with a container when it is present within a receptacle or carrier that also holds the container, e.g., as a package insert. In some instances, the label is used to indicate that the contents are to be used for a specific therapeutic application.

A kit herein can further comprises one or more reagents such as site specific primers or probes to extract, enrich, and/or determine the sequence of the HLA alleles of an individual. The kit may further comprise one or more different HLA alleles. A therapeutic treatment comprises administering to the individual MHC components that have the same HLA alleles as what is found in the individual being treated.

The kit can further comprise one or more other therapeutic agents such as an immune checkpoint inhibitor, an immune checkpoint stimulator, a cancer vaccine, a small molecule therapy, a monoclonal antibody, a cytokine, a cellular therapy, or a combination thereof.

### CERTAIN TERMINOLOGY

The terminology used herein is for the purpose of describing particular cases only and is not intended to be limiting. The below terms are discussed to illustrate meanings of the terms as used in this specification, in addition to the understanding of these terms by those of skill in the art. As used herein and in the appended claims, the singular forms “a”, “an”, and “the” include plural referents unless the context clearly dictates otherwise. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating un-recited number may be a number which, in the context in which it is presented, provides the substantial equivalent of the specifically recited number. Where a range of values is provided, it is understood that each intervening value, to the tenth of the unit of the lower limit unless the context clearly dictates otherwise, between the upper and lower limit of that range and any other stated or intervening value in that stated range, is encompassed within the methods and compositions described herein are. The upper and lower limits of these smaller ranges may independently be included in the smaller ranges and are also encompassed within the methods and compositions described herein, subject to any specifically excluded limit in the stated range. Where the stated range includes one or both of the limits, ranges excluding either or both of those included limits are also included in the methods and compositions described herein.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker). Further, these terms refer to human or animal subjects.

“Treating” or “treatment” refers to both therapeutic treatment and prophylactic or preventative measures, wherein the object is to prevent or slow down (lessen) a targeted pathologic condition or disorder. Those in need of treatment include those already with the disorder, as well as those prone to have the disorder, or those in whom the disorder is to be prevented. For example, a subject or mammal is successfully “treated” for cancer, if, after receiving a therapeutic amount of a subject oligonucleotide conjugate according to the methods of the present disclosure, the subject shows observable and/or measurable reduction in or absence of one or more of the following: reduction in the number of cancer cells or absence of the cancer cells; reduction in the tumor size; inhibition (i.e., slowing to some extent and preferably stopping) of cancer cell infiltration into peripheral organs, including the spread of cancer into soft tissue and bone; inhibition (i.e., slowing to some extent and preferably stopping) of tumor metastasis; inhibition, to some extent, of tumor growth; and/or relief to some extent of one or more of the symptoms associated with the specific cancer; reduced morbidity and/or mortality, and improvement in quality of life issues.

Unless defined otherwise, all technical and scientific terms used herein have the same meaning as commonly understood by one of ordinary skill in the art to which the methods and compositions described herein belong. Although any methods and materials similar or equivalent to those described herein can also be used in the practice or testing of the methods and compositions described herein, representative illustrative methods and materials are now described.

### EXAMPLES

### Example 1

Co-Transfection of Complete Alpha and Beta HLA-DR Chains into Cell Lines that do not Natively Express HLA-DR Resulted in HLA-DR Expression on the Cell Surface.

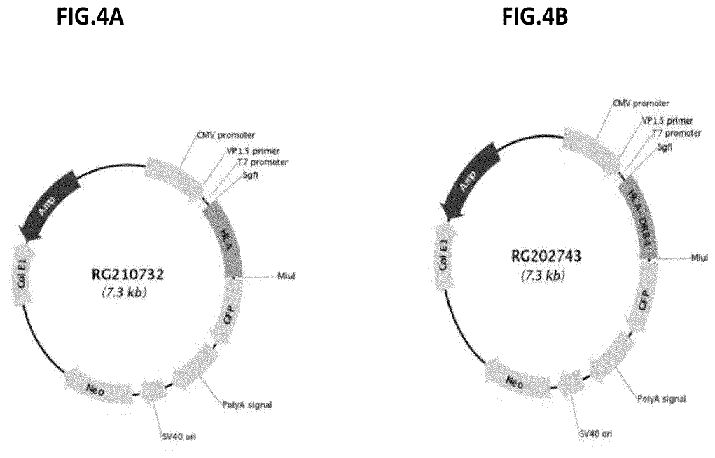

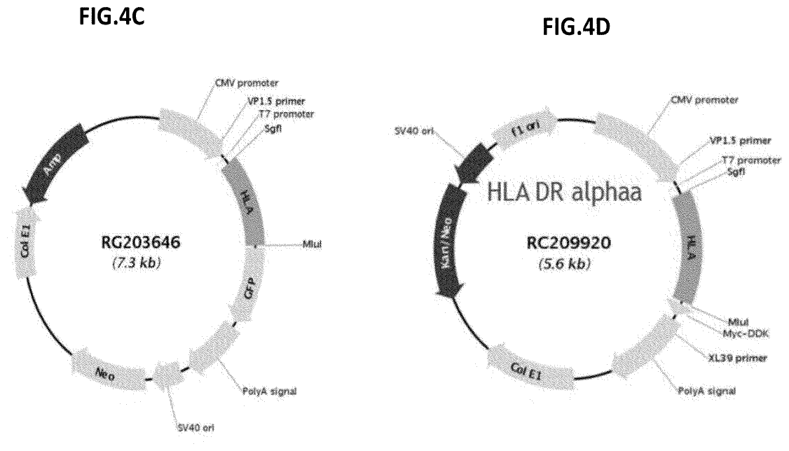

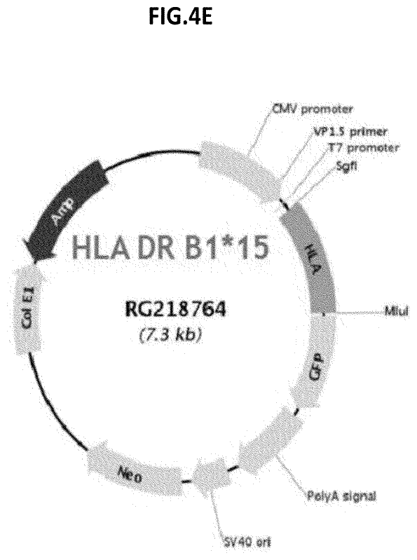

The RKO colonic carcinoma cell line (ATCC CRL-2577), which lack HLA-DR expression, was stably transfected with either HLA-DR A plasmid (alpha, cat #RC209920 (NM_019111)) (FIG. 4D) or HLADRB1*15 plasmid (beta cat #RG218764 (NM_002124)) (FIG. 4F), which were obtained from OnGene Technologies Inc. The RKO cells were also co-transfected with both plasmids. All transfection used electroporation (Using Mirus Bio LLC Kit) according to the manufacture protocol. Transfected cells were subjected to selection pressure using the antibiotic Geneticin® (G418-ThermoFisher) for at least 2 weeks. Transfected cells were tested by FACS using antibodies for HLA-DR A and B (ThermoFisher). For the flow cytometry testing, cells were detached from the flasks and stained with anti HLA-DR alpha (LN3, APC) or HLA-DR beta (UT36, PE) for 30 minutes at 4 degrees C. Further, the transfected cells were washed with FACS buffer twice (PBS with 2% FBS). Cells were then run on a FACS analyzer (CytoFlex S) and data were analyzed using Flowjo software version 10.2.

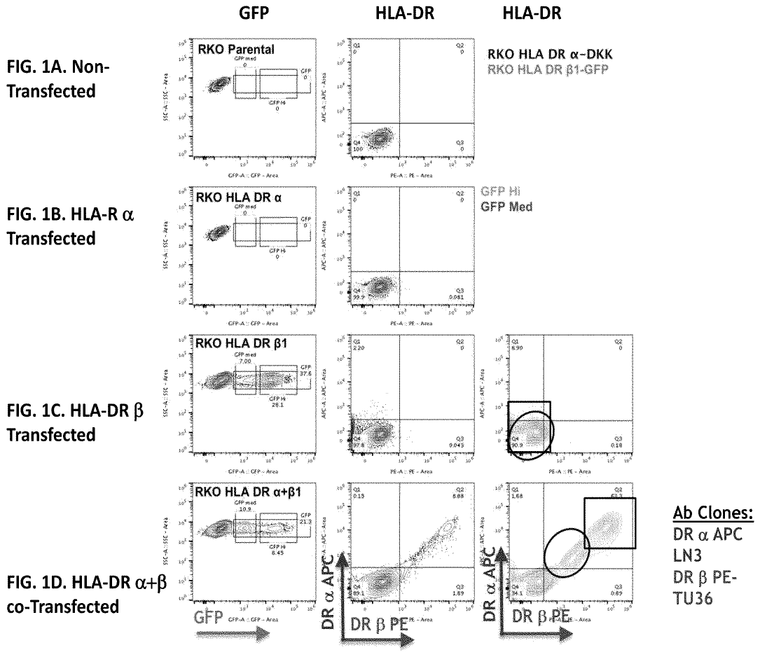

Referring to FIG. 1A, the parental RKO had no surface expression for any HLA receptor. FIG. 1B shows that successful transfection of RKO cells with HLA-DR A (as evidenced by intracellular expression of the Myc-DKK tag; data not shown). However, no HLA-DR was detected expression on the cell surface. Moreover, FIG. 1C shows that in an RKO cell line transfected with HLADR B1 alone, no HLA-DR surface expression was detected even though GFP expression indicated successful transfection. Additionally, FIG. 1D shows surface expression of both alpha and beta chains in cells co-transfected with HLA-DR A and B (transfection confirmed by high and medium GFP expression). This data supports the conclusion that the HLA-DR gene is silent in RKO cells, and surface expression of HLA-DR occurs only when both the A and B1 chains are expressed concurrently.

Further, according to the left column of the FACS plots in FIG. 1A-ID, the large square indicates the GFP positive gated population (i.e. GFP expression) and the small squares indicate the cells expressing medium (dark green overlay displayed in a circle in FIGS. 1C and 1D) and high (light green overlay displayed in a square in FIGS. 1C and 1D) GFP expression. The middle column of the FACS plots indicates surface expression of alpha chain (X axis) and beta chain (Y axis). In the co-transfected cells, both medium and high expression GFP populations present surface expression of HLA DR A and B as seen in the right column of the FACS plots. Overlay and dark green indicated the medium GFP expression population that expresses medium intensity of HLA-DR A and B, and the light green indicated the high GFP expression population with the high HLA-DR A and B expression.

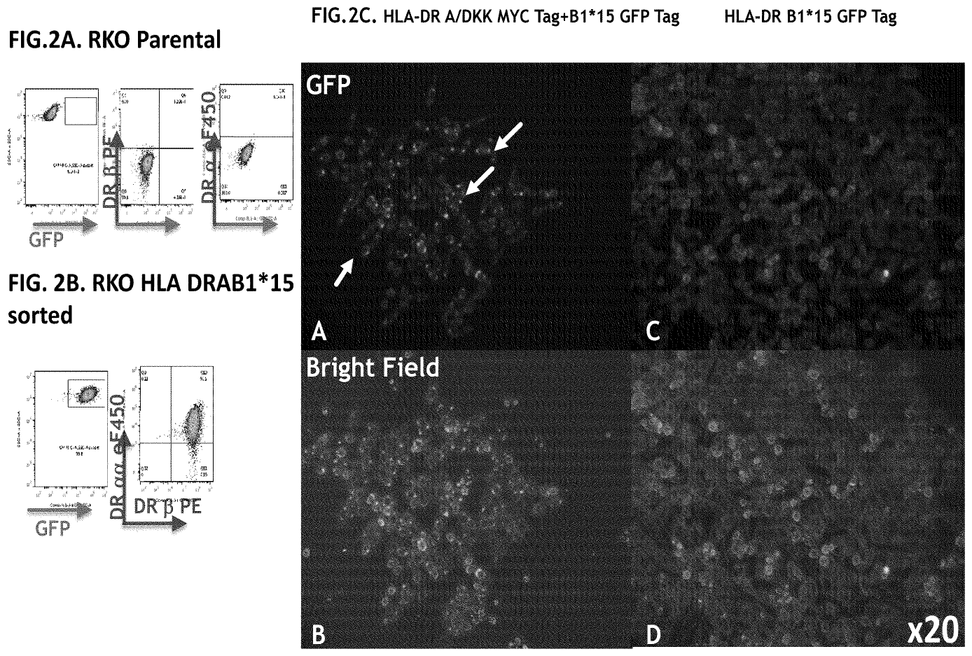

Referring to FIGS. 2A and 2B, the high GFP HLA-DRAB1*15 RKO transfected cell line was sorted (Using Sony Sorter, Sony Biotech) and re-evaluated using flow cytometry analysis (FIG. 2B) in comparison to the parental RKO cell line (FIG. 2A). FIG. 2C shows representative fluorescent pictures (Magnification 20×) of co-transfected GFP HLA-DRAB1*15 RKO cells displayed in the left column (GFP/Bright field) versus GFP HLA-DR B1*15 only in the right column. Co-transfected cells with both alpha and beta units show punctate GFP versus green fluorescent proteins scattered in cytoplasm when only HLA-DR B was transfected. This indicates the association of the alpha and beta chain and migration to the surface of the cells.

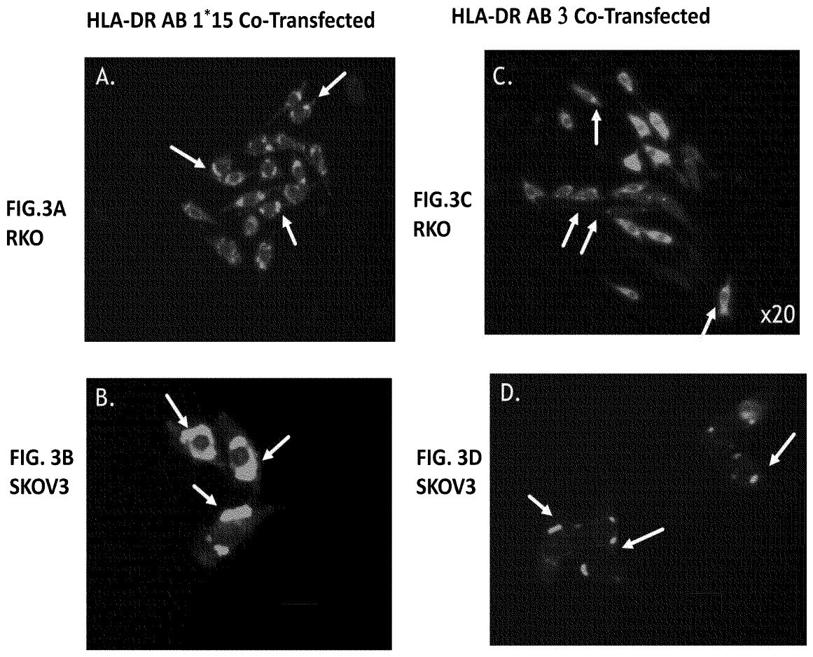

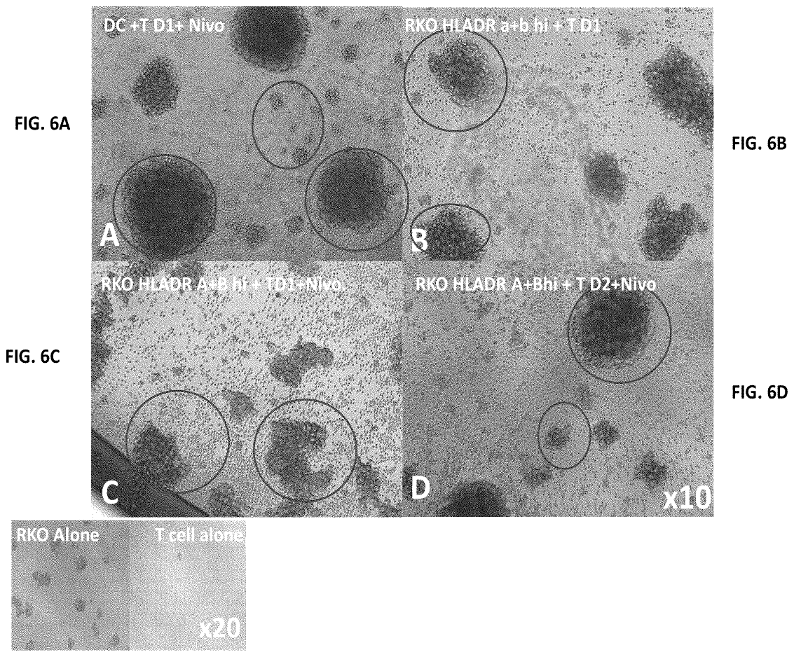

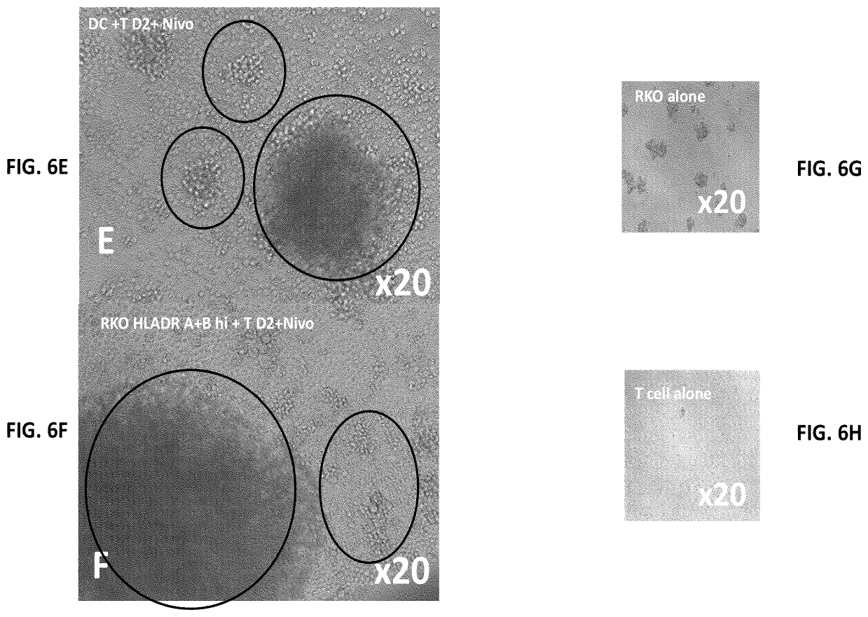

FIGS. 3A-3D show representative fluorescent pictures of stably co-transfected RKO and SKOV3 cells listed as: RKO HLA-DR AB1, SKOV3 HLA-DR AB1, RKO HLA-DR AB3, and SKOV3 HLA-DR AB3. The RKO parental cell line was also co-transfected with HLA-DR A in combination with B3 (RG210732, NM 022555), or B4 (RG202743, NM_021983) or B5 (RG203646, NM_002125), which are all obtained from Origene. Data were confirmed using flow cytometry as described above (data not shown). SKOV3 is an ovarian adenocarcinoma cell line (HTB-7, ATCC), a second cell line that lacks HLA DR expression due to lack of A and B chains expression and was co-transfected with HLA-DR A B1, HLA-DR A B3, HLA-DR A B4 and HLA-DR A B5. The transfected SKOV3 cells were sorted. GFP and HLA-DR expression in RKO cell line and pancreatic adenocarcinoma BxPC3 cell line (CRL-1687, ATCC) was not shown. Plasmids of the different Beta chains are presented in FIG. 4A-4C. Fluorescent pictures of RKO HLA-AB1 and RKO HLA-AB3 are shown in FIGS. 6A and 6C, and fluorescent pictures of SKOV3 HLA-AB1 and SKOV3 HLA-AB3 are shown in FIGS. 6B and 6D. White errors indicate punctuated vesicle expression of GFPs, which indicate the migration of MHC molecules to cell surface.

### Example 2: Proliferation of T Cells is Dependent Upon HLA Expression

Functional mixed lymphocyte reaction, T-cell proliferation, and cytokine release assays are used to test the effect of tumor cells lines expressing HLA-DR in activating T cells compared to non-expressing tumor cells.

### Human Mixed Lymphocyte Reaction Assay

To test whether the co-transfected HLA-DR RKO cells can activate T cells with similar and different HLA-DRs, dendritic cells (DCs) were used as a positive control for the MLR assay. To generate these DCs, the protocol described below was followed:

Human buffy coat was purchased from Stanford Blood Center (Stanford, CA), diluted with PBS, and layered over Ficoll for the isolation of human PBMCs. The human PBMCs were washed 4 times with PBS and cluster of differentiation 14 (CD14+) monocytes were isolated using a human specific CD14+ cell isolation kit with positive selection, as described in the manufacturer's protocol (Miltenyi Biotec, San Diego, CA). CD14+ cells were then seeded at 5×10⁵ cells/mL in complete Roswell Park Memorial Institute (RPMI) 1640 media supplemented with 10% fetal bovine serum (FBS) for 7 days. Cultures were supplemented with recombinant human (rh-) IL-4 (1000 U/mL) (R&D Systems, Minneapolis, MN) and with rh granulocyte-macrophage colony-stimulating factor (GM-CSF) (rh-GMCSF) (500 U/mL) (R&D Systems, Minneapolis, MN) at Days 0, 2 and 5. Immature DCs were harvested, washed, and counted on Day 7.

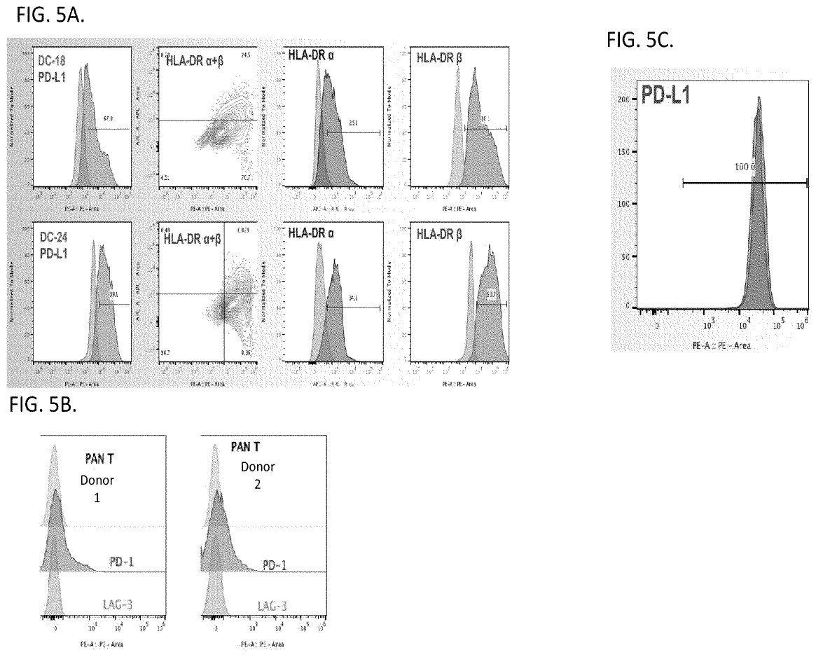

These DCs expressed high HLA-DR and PD-L1 as shown in FIG. 5A of two representative DCs prepared from two different donors (D1 and D2). The sample of each preparation was tested for PD-L1 expression using r-phycoerythrin (RPE) labeled anti-hu-PD-L1 (eBioscience/Affymatrix, Santa Clara, CA) by flow cytometry using a Cytoflex analyzer (Beckman Culture). Further, HLA-DR alpha (APC) and HLA DR beta PE (eBioscience/Affymatrix, Santa Clara, CA) expressions were also evaluated in cells isolated from D1 and D2.

Referring to FIG. 5B, human T Lymphocytes were isolated from buffy coats (Stanford blood Center, CA), diluted with phosphate buffered saline (PBS), and layered over Ficoll for the isolation of PBMCs. The human-PBMCs were washed 4 times with PBS and T lymphocytes and were isolated using a human-specific Pan T-cell isolation kit with negative selection as described in the manufacturer's protocol (Miltenyi Biotec, San Diego, CA). As shown in FIG. 5B, DCs expressed minimal levels of co-inhibitory receptors, such as LAG 3 and PD-1, as expected from rested T cells. Further, referring to FIG. 5C, the parental RKO cell line and HLA-DR AB1*15 co-transfected cells were grown with Eagle's Minimum Essential Medium (MEM) (Corning, Fisher Scientific) with 10% FBS. For stably transfected RKO cells, the G418 was added as a selection antibiotic. Both parental and HLA-DR AB transfected RKO cells expressed high level of PD-L1.

### T-Cell Proliferation Assay & Cytokine Release Assay

The MLR protocol was adapted from Kruisbeek et al, 2004, with some modifications. Primary human-DCs differentiated, HLA-DR AB1 transfected RKO cells, and RKO parental were harvested on the day of experiment for optimal antigen presenting cells status and verified by flow cytometry for high levels of PD-L1 expression and co-stimulatory markers, such as CD80 and CD86, necessary for T-cell activation (data not shown). Cells were counted and treated with a low dose of 50 ug/mL mitomycin C (sigma Aldrich, Saint Louis, MO) to prevent cells from secreting cytokines but functioning only as antigen presentation support to the T cells. Thus, the outcome of the assay was only induced by T cells.

Freshly isolated human-T cells from allogenic donors were harvested following the same protocol described above. T cells were plated with irradiated DCs at a ratio of 10:1 (T: DCs or RKO— for optimal assay conditions) in the presence of different concentrations of anti-PD-1 antibodies (Nivolumab and Pembrolizumab), anti LAG3 antibodies, negative and positive control antibodies, or media alone (to evaluate the baseline reaction). All conditions were plated in 96-well flat bottom tissue culture treated plates (Fisher Scientific Pittsburgh, PA). Cells were cultured using serum free X-vivo15 media (Lonza, Walkersville, MD) to prevent human serum variability between experiments. Cultures were incubated at 37 °C with 5% CO₂ for 5-8 days dependent on different donors. The generation of T cells clumps was monitored under light microscope to catch any indication of T cell proliferation (examples in FIGS. 6A-6F, 7A-7D, and 8A). On the day of harvest, supernatants were collected, and cytokine concentrations were measured using Meso Scale Discovery (MSD LLC., Maryland, MD) kits for IFN-gamma and TNF alpha according to the manufacturer's protocol. For T cells proliferation measurement from MLR assay, T cells were treated with Violet CellTrace™ Violet Cell Proliferation Kit (ThermoFisher, San Diego, CA). On harvest day, cells were stained with anti CD3 antibody PE (ThermoFisher, San Diego, CA). Dead cells (Stained with Lived and dead stain eFlour510, ThermoFisher, San Diego, CA) and GFP positive cells were gated out. CD3 positive cells were gated and analyzed for Violet trace staining.

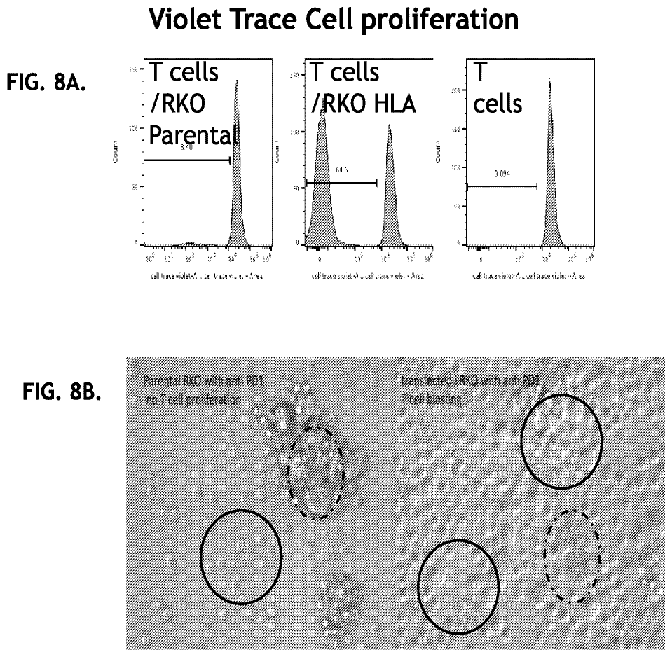

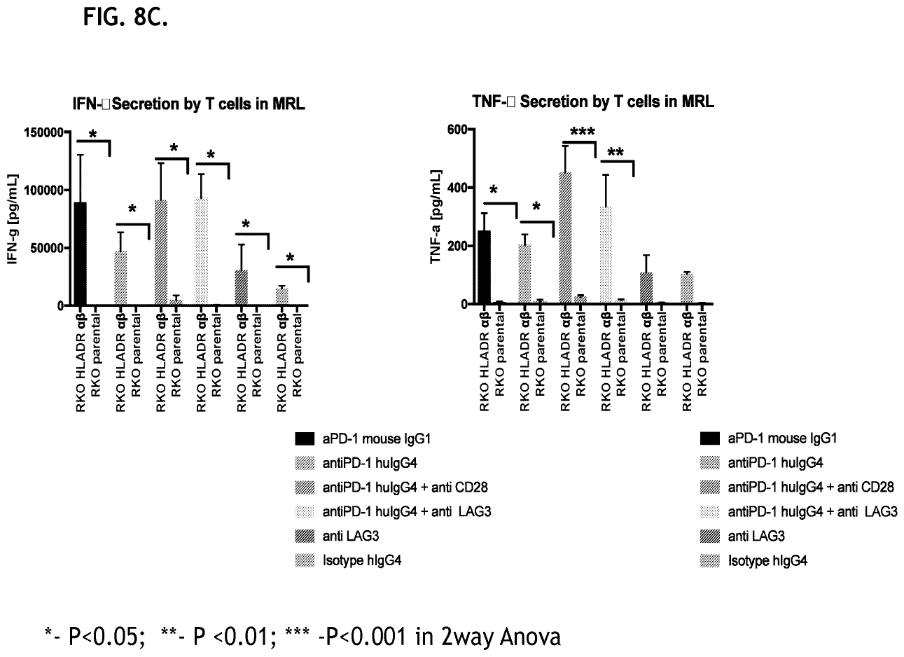

As shown in the middle histogram of FIG. 8A, RKO transfected cells were able to activate the T cells to cause proliferation. T cells that proliferated lost the dye due to equal division of the dye in each proliferation cycle and appeared as negative. When T cells did not proliferate as shown in both the left and right panels of the histograms in FIG. 8A, T cells maintained the dye. DCs showed similar results compared to RKO HLA-DR AB (data not shown). Since RKO and DCs express high level of PD-L1, proliferation in T cells was inhibited due to the expression of PD-L1. As shown in FIG. 8B, addition of anti PD-L1 antibodies increased the proliferation of T cells. Data were acquired using flow cytometry (CytofLEX S analyzer, Beckman Coulter) and data analysis was performed using Flowjo Software Version 10.2.

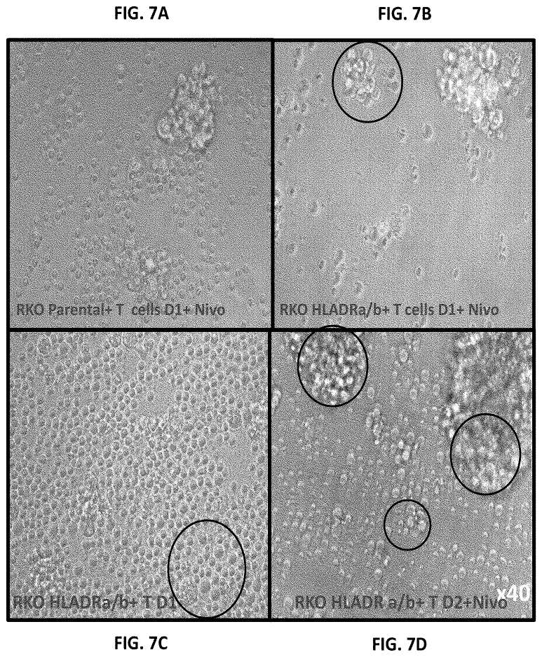

FIGS. 6A-6F and FIGS. 7A-7D showed representative pictures of proliferating T cells obtained from donor 1 and 2 with different magnifications. FIG. 7A demonstrates that donor 1 (D1) T cells were not proliferated when cultured together with RKO parental cells. FIG. 6A shows D1 T cells proliferated after treating with anti PD-1 antibodies and FIG. 6E shows donor 2 (D2) T cells proliferated after treating with anti PD-1 antibodies, respectively. FIGS. 6B and 6C shows D1 T cells proliferated when cultured together with HLA-DR (with both alpha and beta units) transfected RKO cells. Similarly, FIGS. 6D and 6F shows D2 T cells proliferated when cultured together with HLA-DR (with both alpha and beta units) transfected RKO cells. As a contrastF, T cells were not proliferated when T cells were cultured with RKO parental cells (FIG. 6G) or without any treatment (FIG. 6H).

Further, referring to FIGS. 7B-7D, T cells proliferated when cultured together with HLA-DR A+B (with both alpha and beta units) transfected RKO cells. T cell blasts and clusters are shown in circles with solid lines and RKO cells are circled in dashed lines.

Cytokines were measured from the supernatants of the above described cultures using MSD U-Plex Kits (Meso Scale Discovery LLC (Maryland MD). Results were run on MSD MESO QuickPlex SQ 120 analyzer and analyzed using MSD software and GraphPad Prism. For statistical analysis, 2 Way Anova was used. Levels of IFN-gamma, TNF-alpha IL-1beta, and IL-6 were measured. Referring to FIG. 8C, IFN-gamma and TNF-alpha were increased from T cells incubated with RKO HLA-DR cells or DCs (not shown, positive control used as positive control only) when compared to RKO parental line, treatment with checkpoint inhibitors increased the cytokine secretion in these cultures. IL-1beta and IL-6 were not detected or detected at low level indicating that cytokines were secreted due to T cell activation and not from innate cells like DCs or the tumor RKO cells. Data presented in duplicates with SEM. Data from RKO or RKO HLA-DR1 are shown in FIG. 8C and table below.

TABLE 2
Cytokine secretion from T cells activated in MLR with Parental
RKO cell Line versus HLA-DR AB Co-transfected RKO Cell Line.

| | IFN gamma | | | | | | | | |
|---|---|---|---|---|---|---|---|---|---|
| | aPD-1 mouse IgG1 | | | antiPD-1 huIgG4 | | | antiPD-1 huIgG4 + anti CD 28 | | |
| | Mean pg/mL | SEM | N | Mean pg/mL | SEM | N | Mean pg/mL | SEM | N |
| RKO HLADR αβ | 89406.7111 | 41050.7769 | 2 | 46922.14235 | 16630.99135 | 2 | 91365.31965 | 31971.20435 | 2 |
| RKO parental | 344.0823453 | 252.1879897 | 2 | 304.7829054 | 241.6331026 | 2 | 5071.261095 | 3863.607395 | 2 |
| | antiPD-1 huIgG4 + anti LAG3 | | | anti LAG3 | | | Isotype hIgG4 | | |
| | Mean pg/mL | SEM | N | Mean pg/mL | SEM | N | Mean pg/mL | SEM | N |
| RKO HLADR αβ | 93004.7066 | 20627.1864 | 2 | 30500.31218 | 22379.25672 | 2 | 14967.45875 | 2260.14585 | 2 |
| RKO parental | 616.772007 | 198.844166 | 2 | 126.2591885 | 115.3029116 | 2 | 247.4901336 | 243.5212534 | 2 |
| | TNF alpha | | | | | | | | |
| | aPD-1 mouse IgG1 | | | antiPD-1 huIgG4 | | | antiPD-1 huIgG4 + anti CD 28 | | |
| | Mean pg/mL | SEM | N | Mean pg/mL | SEM | N | Mean pg/mL | SEM | N |
| RKO HLADR αβ | 89406.7111 | 41050.7769 | 2 | 46922.14235 | 16630.99135 | 2 | 91365.31965 | 31971.20435 | 2 |
| RKO parental | 344.0823453 | 252.1879897 | 2 | 304.7829054 | 241.6331026 | 2 | 5071.261095 | 3863.607395 | 2 |
| | antiPD-1 huIgG4 + anti LAG3 | | | anti LAG3 | | | Isotype hIgG4 | | |
| | Mean pg/mL | SEM | N | Mean pg/mL | SEM | N | Mean pg/mL | SEM | N |
| RKO HLADR αβ | 93004.7066 | 20627.1864 | 2 | 30500.31218 | 22379.25672 | 2 | 14967.45875 | 2260.14585 | 2 |
| RKO parental | 616.772007 | 198.844166 | 2 | 126.2591885 | 115.3029116 | 2 | 247.4901336 | 243.5212534 | 2 |

### Example 3: Administration of a Non-Naturally Occurring Class I MHC Component

An individual suffering from ovarian cancer is determined to show reduced HLA-A expression in the ovarian cancer relative to baseline HLA-A expression levels in ovarian tissue. The patient is administered an adenoviral vector comprising a non-naturally occurring HLA-A gene modified for enhanced expression in ovarian tissue. Expression of the non-naturally occurring HLA-A gene in the individual is restored.

### Example 4: Targeted Demethylation of Hypermethylated HLA Promoter Regions in a Colon Cancer

An individual suffering from colon cancer previously shown to be unresponsive to immune checkpoint inhibitor therapy has a tumor biopsy. First, expression of each class I HLA and class II HLA gene is determined. Each of the class I HLA genes is shown to have severely reduced expression relative to normal class I HLA expression. DNA from the tumor is extracted as well as DNA from non-cancerous tissue of the same individual. An aliquot of each DNA sample is sequenced for each of HLA-A, HLA-B, and HLA-C genes. The remaining DNA samples are treated with bisulfite and the same genes are subsequently sequenced. Comparison of the non-bisulfite treated sequence with the bisulfite treated DNA reveals that the promoters of each of the three HLA class I genes are methylated with respect to the non-cancerous HLA class I genes at two different CpG sites per promoter.

An immunotherapeutic composition comprising seven different nucleic acid molecules is created, one nucleic acid molecule encodes a deactivated CRISPR-associated nuclease fused to a TET enzyme (a demethylation enzyme) and the remaining six nucleic acid molecules encode guide RNA (gRNA), each gRNA targeted one of the six methylated CpG sites identified in the promoters. The composition is administered to the individual. Expression of class I HLA molecules in the individual is assessed one day later and shown to have risen. An immune checkpoint inhibitor therapy is then administered to the individual.

### Example 5: Administration of a Class II MHC Component

An individual suffering from pancreatic cancer is administered a liposome comprising a plasmid encoding the HLA-DQA1 and HLA-DQB1 genes.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

TABLE 3
HLA alleles

| | | | | | | | | | | |
|---|---|---|---|---|---|---|---|---|---|---|
| 1 | A*01 | A*02 | A*03 | A*11 | A*23 | A*24 | A*25 | A*26 | A*29 | A*30 |
| 2 | A*31 | A*32 | A*33 | A*34 | A*36 | A*43 | A*66 | A*68 | A*69 | A*74 |
| 3 | A*80 | B*07 | B*08 | B*13 | B*14 | B*15 | B*18 | B*27 | B*35 | B*37 |
| 4 | B*38 | B*39 | B*40 | B*41 | B*42 | B*44 | B*45 | B*46 | B*47 | B*48 |
| 5 | B*49 | B*50 | B*51 | B*52 | B*53 | B*54 | B*55 | B*56 | B*57 | B*58 |
| 6 | B*59 | B*67 | B*73 | B*78 | B*81 | B*82 | B*83 | C*01 | C*02 | C*03 |
| 7 | C*04 | C*05 | C*06 | C*07 | C*08 | C*12 | C*14 | C*15 | C*16 | C*17 |
| 8 | C*18 | E*01 | F*01 | G*01 | H*01 | H*02 | H*03 | J*01 | J*02 | K*01 |
| 9 | L*01 | N*01 | P*01 | P*02 | S*01 | T*01 | T*02 | T*03 | U*01 | V*01 |
| 10 | W*01 | W*02 | W*03 | W*04 | W*05 | Y*01 | Y*02 | Y*03 | DRA*01 | DQA1*01 |
| 11 | DQA1*02 | DQA1*03 | DQA1*04 | DQA1*05 | DQA1*06 | DQB1*02 | DQB1*03 | DQB1*04 | DQB1*05 | DQB1*06 |
| 12 | DPA1*01 | DPA1*02 | DPA1*03 | DPA1*04 | DPA2*01 | DPA2*02 | DPB1*01 | DPB1*02 | DPB1*03 | DPB1*04 |
| 13 | DPB1*05 | DPB1*06 | DPB1*08 | DPB1*09 | DPB1*10 | DPB1*100 | DPB1*101 | DPB1*102 | DPB1*103 | DPB1*104 |
| 14 | DPB1*105 | DPB1*106 | DPB1*107 | DPB1*108 | DPB1*109 | DPB1*11 | DPB1*110 | DPB1*111 | DPB1*112 | DPB1*113 |
| 15 | DPB1*114 | DPB1*115 | DPB1*116 | DPB1*117 | DPB1*118 | DPB1*119 | DPB1*120 | DPB1*121 | DPB1*122 | DPB1*123 |
| 16 | DPB1*124 | DPB1*125 | DPB1*126 | DPB1*127 | DPB1*128 | DPB1*129 | DPB1*13 | DPB1*130 | DPB1*131 | DPB1*132 |
| 17 | DPB1*133 | DPB1*134 | DPB1*135 | DPB1*136 | DPB1*137 | DPB1*138 | DPB1*139 | DPB1*14 | DPB1*140 | DPB1*141 |
| 18 | DPB1*142 | DPB1*143 | DPB1*144 | DPB1*145 | DPB1*146 | DPB1*147 | DPB1*148 | DPB1*149 | DPB1*15 | DPB1*150 |
| 19 | DPB1*151 | DPB1*152 | DPB1*153 | DPB1*154 | DPB1*155 | DPB1*156 | DPB1*157 | DPB1*158 | DPB1*159 | DPB1*16 |
| 20 | DPB1*160 | DPB1*161 | DPB1*162 | DPB1*163 | DPB1*164 | DPB1*165 | DPB1*166 | DPB1*167 | DPB1*168 | DPB1*169 |
| 21 | DPB1*17 | DPB1*170 | DPB1*171 | DPB1*172 | DPB1*173 | DPB1*174 | DPB1*175 | DPB1*176 | DPB1*177 | DPB1*178 |
| 22 | DPB1*179 | DPB1*18 | DPB1*180 | DPB1*181 | DPB1*182 | DPB1*183 | DPB1*184 | DPB1*185 | DPB1*186 | DPB1*187 |
| 23 | DPB1*188 | DPB1*189 | DPB1*19 | DPB1*190 | DPB1*191 | DPB1*192 | DPB1*193 | DPB1*194 | DPB1*195 | DPB1*196 |
| 24 | DPB1*197 | DPB1*198 | DPB1*199 | DPB1*20 | DPB1*200 | DPB1*201 | DPB1*202 | DPB1*203 | DPB1*204 | DPB1*205 |
| 25 | DPB1*206 | DPB1*207 | DPB1*208 | DPB1*209 | DPB1*21 | DPB1*210 | DPB1*211 | DPB1*212 | DPB1*213 | DPB1*214 |
| 26 | DPB1*215 | DPB1*216 | DPB1*217 | DPB1*218 | DPB1*219 | DPB1*22 | DPB1*220 | DPB1*221 | DPB1*222 | DPB1*223 |
| 27 | DPB1*224 | DPB1*225 | DPB1*226 | DPB1*227 | DPB1*228 | DPB1*229 | DPB1*23 | DPB1*230 | DPB1*231 | DPB1*232 |
| 28 | DPB1*233 | DPB1*234 | DPB1*235 | DPB1*236 | DPB1*237 | DPB1*238 | DPB1*239 | DPB1*24 | DPB1*240 | DPB1*241 |
| 29 | DPB1*242 | DPB1*243 | DPB1*244 | DPB1*245 | DPB1*246 | DPB1*247 | DPB1*248 | DPB1*249 | DPB1*25 | DPB1*250 |
| 30 | DPB1*251 | DPB1*252 | DPB1*253 | DPB1*254 | DPB1*255 | DPB1*256 | DPB1*257 | DPB1*258 | DPB1*259 | DPB1*26 |
| 31 | DPB1*260 | DPB1*261 | DPB1*262 | DPB1*263 | DPB1*264 | DPB1*265 | DPB1*266 | DPB1*267 | DPB1*268 | DPB1*269 |
| 32 | DPB1*27 | DPB1*270 | DPB1*271 | DPB1*272 | DPB1*273 | DPB1*274 | DPB1*275 | DPB1*276 | DPB1*277 | DPB1*278 |
| 33 | DPB1*279 | DPB1*28 | DPB1*280 | DPB1*281 | DPB1*282 | DPB1*283 | DPB1*284 | DPB1*285 | DPB1*286 | DPB1*287 |
| 34 | DPB1*288 | DPB1*289 | DPB1*29 | DPB1*290 | DPB1*291 | DPB1*292 | DPB1*293 | DPB1*294 | DPB1*295 | DPB1*296 |
| 35 | DPB1*297 | DPB1*298 | DPB1*299 | DPB1*30 | DPB1*300 | DPB1*301 | DPB1*302 | DPB1*303 | DPB1*304 | DPB1*305 |
| 36 | DPB1*306 | DPB1*307 | DPB1*308 | DPB1*309 | DPB1*31 | DPB1*310 | DPB1*311 | DPB1*312 | DPB1*313 | DPB1*314 |
| 37 | DPB1*315 | DPB1*316 | DPB1*317 | DPB1*318 | DPB1*319 | DPB1*32 | DPB1*320 | DPB1*321 | DPB1*322 | DPB1*323 |
| 38 | DPB1*324 | DPB1*325 | DPB1*326 | DPB1*327 | DPB1*328 | DPB1*329 | DPB1*33 | DPB1*330 | DPB1*331 | DPB1*332 |
| 39 | DPB1*333 | DPB1*334 | DPB1*335 | DPB1*336 | DPB1*337 | DPB1*338 | DPB1*339 | DPB1*34 | DPB1*340 | DPB1*341 |
| 40 | DPB1*342 | DPB1*343 | DPB1*344 | DPB1*345 | DPB1*346 | DPB1*347 | DPB1*348 | DPB1*349 | DPB1*35 | DPB1*350 |
| 41 | DPB1*351 | DPB1*352 | DPB1*353 | DPB1*354 | DPB1*355 | DPB1*356 | DPB1*357 | DPB1*358 | DPB1*359 | DPB1*36 |
| 42 | DPB1*360 | DPB1*361 | DPB1*362 | DPB1*363 | DPB1*364 | DPB1*365 | DPB1*366 | DPB1*367 | DPB1*368 | DPB1*369 |
| 43 | DPB1*37 | DPB1*370 | DPB1*371 | DPB1*372 | DPB1*373 | DPB1*374 | DPB1*375 | DPB1*376 | DPB1*377 | DPB1*378 |
| 44 | DPB1*379 | DPB1*38 | DPB1*380 | DPB1*381 | DPB1*382 | DPB1*383 | DPB1*384 | DPB1*385 | DPB1*386 | DPB1*387 |
| 45 | DPB1*388 | DPB1*389 | DPB1*39 | DPB1*390 | DPB1*391 | DPB1*392 | DPB1*393 | DPB1*394 | DPB1*395 | DPB1*396 |
| 46 | DPB1*397 | DPB1*398 | DPB1*399 | DPB1*40 | DPB1*400 | DPB1*401 | DPB1*402 | DPB1*403 | DPB1*404 | DPB1*405 |
| 47 | DPB1*406 | DPB1*407 | DPB1*408 | DPB1*409 | DPB1*41 | DPB1*410 | DPB1*411 | DPB1*412 | DPB1*413 | DPB1*414 |
| 48 | DPB1*415 | DPB1*416 | DPB1*417 | DPB1*418 | DPB1*419 | DPB1*420 | DPB1*421 | DPB1*422 | DPB1*423 | DPB1*424 |
| 49 | DPB1*425 | DPB1*426 | DPB1*427 | DPB1*428 | DPB1*429 | DPB1*430 | DPB1*431 | DPB1*432 | DPB1*433 | DPB1*434 |
| 50 | DPB1*435 | DPB1*436 | DPB1*437 | DPB1*438 | DPB1*439 | DPB1*44 | DPB1*440 | DPB1*441 | DPB1*442 | DPB1*443 |
| 51 | DPB1*444 | DPB1*445 | DPB1*446 | DPB1*447 | DPB1*448 | DPB1*449 | DPB1*45 | DPB1*450 | DPB1*451 | DPB1*452 |
| 52 | DPB1*453 | DPB1*454 | DPB1*455 | DPB1*456 | DPB1*457 | DPB1*458 | DPB1*459 | DPB1*46 | DPB1*460 | DPB1*461 |
| 53 | DPB1*462 | DPB1*463 | DPB1*464 | DPB1*465 | DPB1*466 | DPB1*467 | DPB1*468 | DPB1*469 | DPB1*47 | DPB1*470 |
| 54 | DPB1*471 | DPB1*472 | DPB1*473 | DPB1*474 | DPB1*475 | DPB1*476 | DPB1*477 | DPB1*478 | DPB1*479 | DPB1*48 |
| 55 | DPB1*480 | DPB1*481 | DPB1*482 | DPB1*483 | DPB1*484 | DPB1*485 | DPB1*486 | DPB1*487 | DPB1*488 | DPB1*489 |
| 56 | DPB1*49 | DPB1*490 | DPB1*491 | DPB1*492 | DPB1*493 | DPB1*494 | DPB1*495 | DPB1*496 | DPB1*497 | DPB1*498 |
| 57 | DPB1*499 | DPB1*50 | DPB1*500 | DPB1*501 | DPB1*502 | DPB1*503 | DPB1*504 | DPB1*505 | DPB1*506 | DPB1*507 |
| 58 | DPB1*508 | DPB1*509 | DPB1*51 | DPB1*510 | DPB1*511 | DPB1*512 | DPB1*513 | DPB1*514 | DPB1*515 | DPB1*516 |
| 59 | DPB1*517 | DPB1*518 | DPB1*519 | DPB1*52 | DPB1*520 | DPB1*521 | DPB1*522 | DPB1*523 | DPB1*524 | DPB1*525 |
| 60 | DPB1*526 | DPB1*527 | DPB1*528 | DPB1*529 | DPB1*53 | DPB1*530 | DPB1*531 | DPB1*532 | DPB1*533 | DPB1*534 |
| 61 | DPB1*535 | DPB1*536 | DPB1*537 | DPB1*538 | DPB1*539 | DPB1*54 | DPB1*540 | DPB1*541 | DPB1*542 | DPB1*543 |
| 62 | DPB1*544 | DPB1*545 | DPB1*546 | DPB1*547 | DPB1*548 | DPB1*549 | DPB1*55 | DPB1*550 | DPB1*551 | DPB1*552 |
| 63 | DPB1*553 | DPB1*554 | DPB1*555 | DPB1*556 | DPB1*557 | DPB1*558 | DPB1*559 | DPB1*56 | DPB1*560 | DPB1*561 |
| 64 | DPB1*562 | DPB1*563 | DPB1*564 | DPB1*565 | DPB1*566 | DPB1*567 | DPB1*568 | DPB1*569 | DPB1*57 | DPB1*570 |
| 65 | DPB1*571 | DPB1*572 | DPB1*573 | DPB1*574 | DPB1*575 | DPB1*576 | DPB1*577 | DPB1*578 | DPB1*579 | DPB1*58 |
| 66 | DPB1*580 | DPB1*581 | DPB1*582 | DPB1*583 | DPB1*584 | DPB1*585 | DPB1*586 | DPB1*587 | DPB1*588 | DPB1*589 |
| 67 | DPB1*59 | DPB1*590 | DPB1*591 | DPB1*592 | DPB1*593 | DPB1*594 | DPB1*595 | DPB1*596 | DPB1*597 | DPB1*598 |
| 68 | DPB1*599 | DPB1*60 | DPB1*600 | DPB1*601 | DPB1*602 | DPB1*603 | DPB1*604 | DPB1*605 | DPB1*606 | DPB1*607 |
| 69 | DPB1*608 | DPB1*609 | DPB1*61 | DPB1*610 | DPB1*611 | DPB1*612 | DPB1*613 | DPB1*614 | DPB1*615 | DPB1*616 |
| 70 | DPB1*617 | DPB1*618 | DPB1*619 | DPB1*62 | DPB1*620 | DPB1*621 | DPB1*622 | DPB1*623 | DPB1*624 | DPB1*625 |
| 71 | DPB1*626 | DPB1*627 | DPB1*628 | DPB1*629 | DPB1*63 | DPB1*630 | DPB1*631 | DPB1*632 | DPB1*633 | DPB1*634 |
| 72 | DPB1*635 | DPB1*636 | DPB1*637 | DPB1*638 | DPB1*639 | DPB1*64 | DPB1*640 | DPB1*641 | DPB1*642 | DPB1*643 |
| 73 | DPB1*644 | DPB1*645 | DPB1*646 | DPB1*647 | DPB1*648 | DPB1*649 | DPB1*65 | DPB1*650 | DPB1*651 | DPB1*652 |
| 74 | DPB1*653 | DPB1*654 | DPB1*655 | DPB1*656 | DPB1*657 | DPB1*658 | DPB1*659 | DPB1*66 | DPB1*660 | DPB1*661 |
| 75 | DPB1*662 | DPB1*663 | DPB1*664 | DPB1*665 | DPB1*666 | DPB1*667 | DPB1*668 | DPB1*669 | DPB1*67 | DPB1*670 |
| 76 | DPB1*671 | DPB1*672 | DPB1*673 | DPB1*674 | DPB1*675 | DPB1*676 | DPB1*677 | DPB1*678 | DPB1*679 | DPB1*68 |
| 77 | DPB1*680 | DPB1*681 | DPB1*682 | DPB1*683 | DPB1*684 | DPB1*685 | DPB1*686 | DPB1*687 | DPB1*688 | DPB1*689 |
| 78 | DPB1*69 | DPB1*690 | DPB1*691 | DPB1*692 | DPB1*693 | DPB1*694 | DPB1*695 | DPB1*696 | DPB1*697 | DPB1*698 |
| 79 | DPB1*699 | DPB1*70 | DPB1*700 | DPB1*701 | DPB1*702 | DPB1*703 | DPB1*704 | DPB1*705 | DPB1*706 | DPB1*707 |
| 80 | DPB1*708 | DPB1*709 | DPB1*71 | DPB1*710 | DPB1*711 | DPB1*712 | DPB1*713 | DPB1*714 | DPB1*715 | DPB1*716 |
| 81 | DPB1*717 | DPB1*718 | DPB1*719 | DPB1*72 | DPB1*720 | DPB1*721 | DPB1*722 | DPB1*723 | DPB1*724 | DPB1*725 |
| 82 | DPB1*726 | DPB1*727 | DPB1*728 | DPB1*729 | DPB1*73 | DPB1*730 | DPB1*731 | DPB1*732 | DPB1*733 | DPB1*734 |
| 83 | DPB1*735 | DPB1*736 | DPB1*737 | DPB1*738 | DPB1*739 | DPB1*74 | DPB1*740 | DPB1*741 | DPB1*742 | DPB1*743 |
| 84 | DPB1*744 | DPB1*745 | DPB1*746 | DPB1*747 | DPB1*748 | DPB1*749 | DPB1*75 | DPB1*750 | DPB1*751 | DPB1*752 |
| 85 | DPB1*753 | DPB1*754 | DPB1*755 | DPB1*756 | DPB1*757 | DPB1*758 | DPB1*759 | DPB1*76 | DPB1*760 | DPB1*761 |
| 86 | DPB1*762 | DPB1*763 | DPB1*77 | DPB1*78 | DPB1*79 | DPB1*80 | DPB1*81 | DPB1*82 | DPB1*83 | DPB1*84 |
| 87 | DPB1*85 | DPB1*86 | DPB1*87 | DPB1*88 | DPB1*89 | DPB1*90 | DPB1*91 | DPB1*92 | DPB1*93 | DPB1*94 |
| 88 | DPB1*95 | DPB1*96 | DPB1*97 | DPB1*98 | DPB1*99 | DPB2*01 | DPB2*02 | DPB2*03 | DMA*01 | DMB*01 |
| 89 | DOA*01 | DOB*01 | DRB1*01 | DRB1*03 | DRB1*04 | DRB1*07 | DRB1*08 | DRB1*09 | DRB1*10 | DRB1*11 |
| 90 | DRB1*12 | DRB1*13 | DRB1*14 | DRB1*15 | DRB1*16 | DRB2*01 | DRB3*01 | DRB3*02 | DRB3*03 | DRB4*01 |
| 91 | DRB4*02 | DRB4*03 | DRB5*01 | DRB5*02 | DRB6*01 | DRB6*02 | DRB7*01 | DRB8*01 | DRB9*01 | HFE*001 |
| 92 | MICA*002 | MICA*007 | MICA*008 | MICA*009 | MICA*010 | MICA*012 | MICA*018 | MICA*019 | MICB*002 | MICB*004 |
| 93 | MICB*005 | TAP1*01 | TAP1*02 | TAP1*03 | TAP1*04 | TAP1*05 | TAP1*06 | TAP2*01 | TAP2*02 | |

1.-160. (canceled)

161. An immunotherapeutic composition comprising:
(i) a first nucleic acid molecule encoding one or more regulators of a major histocompatibility complex (MHC) component or a functional fragment thereof for use in treating a cancer in a subject, wherein the one or more regulators is a NOD-like receptor family CARD domain containing 5 (NLRC5), a member of a regulatory factor X (RFX) family, or a nuclear transcription factor Y (NF-Y); and
(ii) a second nucleic acid molecule encoding a major histocompatibility complex (MHC) class II transactivator (CIITA).

162. The immunotherapeutic composition for use according to claim 161, wherein the cancer is an ovarian cancer, a pancreatic cancer, a colon cancer, a breast cancer, a prostate cancer, a lung cancer, a liver cancer, a leukemia, a lymphoma, a myeloma, a bladder cancer, a cervical cancer, an endometrial cancer, a lung cancer, a bronchus cancer, a colon and rectal cancer, stomach cancer, gastric cancer, gall bladder cancer, gastrointestinal stromal tumor cancer, thyroid cancer, head and neck cancer, oropharyngeal cancer, esophageal cancer, a melanoma, a non-melanoma skin cancer, a Merkel cell carcinoma, a virally-induced cancer, a neuroblastoma, a renal cancer, a renal cell cancer, a renal pelvis cancer, a sarcoma, a glioma, a brain tumor, or a carcinoma.

163. The immunotherapeutic composition for use according to claim 161, wherein the cancer has reduced MHC expression.

164. The immunotherapeutic composition for use according to claim 161, wherein the immunotherapeutic composition is formulated for administration to the subject with Preliminary Amendment and Response to the Notice to File Missing Parts an additional therapeutic compound that comprises an immune checkpoint inhibitor.

165. The immunotherapeutic composition for use according to claim 164, wherein the immune checkpoint inhibitor is a molecule which binds to A2AR, B7-H3, B7-H4, BTLA, CTLA-4, IDO, KIR, LAG3, PD-1, TIM-3, VISTA, or a ligand thereof.

166. The immunotherapeutic composition for use according to claim 161, wherein the immunotherapeutic composition is formulated for administration to the subject with an additional therapeutic compound that comprises an immune checkpoint stimulator.

167. The immunotherapeutic composition for use according to claim 166, wherein the immune checkpoint stimulator is a molecule which binds to CD27, CD28, CD40, CD122, CD137, OX40, GITR, ICOS, or a ligand thereof.

168. The immunotherapeutic composition for use according to claim 161, wherein the immunotherapeutic composition is formulated for administration to the subject with an additional therapeutic compound that comprises a cancer vaccine.

169. The immunotherapeutic composition for use according to claim 161, wherein the immunotherapeutic composition is formulated for administration to the subject with an additional therapeutic compound that comprises a small molecule therapy.

170. The immunotherapeutic composition for use according to claim 161, wherein the immunotherapeutic composition is formulated for administration to the subject with an additional therapeutic compound that comprises a monoclonal antibody.

171. The immunotherapeutic composition for use according to claim 161, wherein the immunotherapeutic composition is formulated for administration to the subject with an additional therapeutic compound that comprises a cytokine.

172. The immunotherapeutic composition for use according to claim 161, wherein the immunotherapeutic composition is formulated for administration to the subject with an additional therapeutic compound that comprises a cellular therapy.

173. The immunotherapeutic composition for use of according claim 161, wherein the first nucleic acid molecule is a deoxyribose nucleic acid (DNA).

174. The immunotherapeutic composition for use of according claim 161, wherein the first nucleic acid molecule is a ribonucleic acid (RNA).

175. The immunotherapeutic composition for use of according claim 161, wherein the second nucleic acid molecule is a deoxyribose nucleic acid (DNA).

176. The immunotherapeutic composition for use of according claim 161, wherein the second nucleic acid molecule is a ribonucleic acid (RNA).

177. The immunotherapeutic composition for use according to claim 161, wherein the first nucleic acid molecule is a plasmid.

178. The immunotherapeutic composition for use according to claim 161, wherein the first nucleic acid molecule is a viral vector.

179. The immunotherapeutic composition for use of claim 178, wherein the viral vector is an alphavirus, a retrovirus, an adenovirus, a herpes virus, a poxvirus, a lentivirus, an oncolytic virus, a reovirus, or an adeno associated virus (AAV).

180. The immunotherapeutic composition for use according to claim 161, wherein the first nucleic acid molecule and the second nucleic acid molecule are formulated in a liposome, an exosome, a lipid nanoparticle, or a biomaterial.
